(19) United States
(12) Patent Application Publication
(10) Pub. No.: US 2010/0105657 A1
(43) Pub. Date: Apr. 29, 2010

(54) Compounds, Process for their Preparation, Intermediates, Pharmaceutical Compositions and their use in the Treatment of 5-HT6 Mediated Disorders such as Alzheimer's Disease, Cognitive Disorders, Cognitive Impairment Associated with Schizophrenia, Obesity and Parkinson's Disease

(75) Inventors: Gunnar Nordvall, Sodertalje (SE); Carl Petersson, Sodertalje (SE); Fernando Sehgelmeble, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/993,377

(22) PCT Filed: Jul. 3, 2006
(86) PCT No.: PCT/SE2006/000827

§ 371 Date: Dec. 20, 2007

(30) Foreign Application Priority Data
Jul. 5, 2005 (SE) 0501579-7

(51) Int. Cl. A61K 31/553; C07D 267/14; C07D 223/16; A61K 31/55; A61P 25/28

(52) U.S. Cl. 514/211.09; 540/552; 540/593; 514/213.01

(57) ABSTRACT

The present invention relates to new compounds of formula (I), or salts, solvates or solvated salts thereof, process for their preparation and to new intermediates used in the preparation thereof, pharmaceutical compositions containing said compounds and to the use of said compounds in the treatment of 5-HT6 mediated disorders such as Alzheimer's disease, cognitive disorders, cognitive impairment associated with schizophrenia, obesity and Parkinson's disease.

### FIELD OF THE INVENTION

The present invention relates to new compounds, to pharmaceutical compositions containing said compounds and to the use of said compounds in therapy. The present invention further relates to processes for the preparation of said compounds and to new intermediates and their use in the preparation of the new compounds.

### BACKGROUND OF THE INVENTION

Serotonin (5-hydroxy-tryptamine) (5-HT) receptors play an important role in many physiological and pathological functions like anxiety, sleep regulation, aggression, feeding and depression. The 5-HT receptors are distributed throughout the body and can be divided into seven different 5-HT receptor subtypes, i.e. 5-HT1-5-HT7, with different properties. The 5-HT6 receptor is mostly found in the central nervous system (CNS). From in situ hybridization studies it is known that the 5-HT6 receptor in rat brain is localized in areas like striatum, nucleus accumbens, olfactory tubercle and hippocampal formation (Ward et al., Neuroscience, 64, p 1105-1111, 1995).

Scientific research has revealed a potential therapeutic use for modulators of the 5-HT6 receptor, especially with regard to various CNS disorders. Blocking 5-HT6 receptor function has been shown to enhance cholinergic transmission (Bentley et al, Br J Pharmacol 126: 1537-1542, 1999; Riemer et al J Med Chem 46, 1273-1276). 5-HT6 antagonist have also been shown to reverse cognitive deficits in in vivo cognition models induced by the muscarinic antagonist scopolamine (Woolley et al. Phychopharmacolgy, 170, 358-367, 2003; Foley et al. Neuropsychopharmacology, 29 93-100, 2004)

Studies have shown that 5-HT6 antagonists increase levels of glutamate and aspartate in the frontal cortex and dorsal hippocampus as well as acetylcholine in the frontal cortex. These neurochemicals are known to be involved in memory and cognition (Dawson et al., Neuropsychopharmacology., 25(5), p 662-668, 2001) (Gerard et al., Brain Res., 746, p 207-219, 1997) (Riemer et al J Med Chem 46(7), p 1273-1276, 2003).

Acetylcholinesterase inhibitors increase the levels of acetylcholine in the CNS and are used in the treatment of cognitive disorders such as Alzheimer's disease. 5-HT6 antagonists may therefore be used in the treatment of cognitive disorders.

Studies have also shown that 5-HT6 antagonist increases the level of dopamine and noradrenaline in the medial prefrontal cortex (Lacroix et al. Synapse 51, 158-164, 2004). In addition, 5-HT6 receptor antagonists have been shown to improve performance in the attentional set shifting task (Hatcher et al. Psychopharmacology 181(2):253-9, 2005). Therefore, 5-HT6 ligands are expected to be useful in the treatment of disorders where cognitive deficits are a feature, such as schizophrenia. Several antidepressants and atypical antipsychotics bind to the 5-HT6 receptor and this may be a factor in their profile of activities (Roth et al., J. Pharm. Exp. Therapeut., 268, 1402-1420, 1994; Sleight et al., Exp. Opin. Ther. Patents, 8, 1217-1224, 1998; Kohen et al., J. Neurochem., 66(1), p 47-56, 1996; Sleight et al. Brit. J. Pharmacol., 124, p 556-562, 1998; Bourson et al., Brit. J. Pharmacol., 125, p 1562-1566, 1998).

Stean et al., (Brit. J. Pharmacol. 127 Proc. Supplement 131P, 1999) have described the potential use of 5-HT6 modulators in the treatment of epilepsy. 5-HT6 receptors have also been linked to generalized stress and anxiety states (Yoshioka et al., Life Sciences, 62, 17/18, p 1473-1477, 1998). 5-HT6 agonists have been shown to elevate levels of GABA in brain regions associated with anxiety and shown positive effects in models predictive of obsessive-compulsive disorder (Schechter et a. NeuroRx. 2005 October; 2(4): 590-611). The use of modulators for this receptor is therefore expected for a wide range of CNS disorders.

Pullagurla et al (Pharmacol Biochem Behay. 78(2):263-8, 2004) have described the potential use of 5-11T6 antagonists in disorders were the dopamine transmission is affected, for example a combination between a 5-HT6 antagonist and a dopamine enhancer for example levodopa/carbidopa or amantidine would be expected to have advantages compared to administration of only a dopamine enhancer.

Moreover, a reduction in food intake in rats has been reported using 5-HT6 receptor modulators (Bentley et al., Br. J. Pharmacol. Suppl. 126, P66, 1999; Bentley et al. J. Psychopharmacol. Supl. A64, 255, 1997; Pendharkar et al Society for Neuroscience, 2005). 5-HT6 receptor modulators may therefore also be useful in the treatment of feeding disorders like anorexia, obesity, bulimia and similar disorders and also type 2 diabetes.

### DETAILED DESCRIPTION OF THE INVENTION

The object of the present invention is to provide compounds exhibiting a modulating activity at the 5-hydroxy-tryptamine 6 receptor.

The present invention provides compounds of formula I

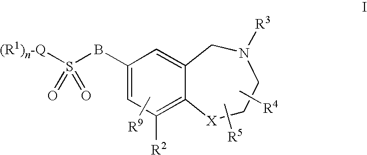

wherein:

Q is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl or C₁₋₁₀alkyl;

R¹ is hydrogen, hydroxyl, halogen, C₁₋₁₀alkyl, C₂₋₁₀allcenyl, C₂₋₁₀alkynyl, C₁₋₁₀alkoxy, N(R¹⁰)₂, C₆₋₁₀arylC₀₋₆alkyl, C₅₋₆heteroarylC₀₋₆alkyl, C₁₋₆haloalkyl, C₁₋₆haloalkylO, R⁸OC₀₋₆alkyl, CN, SR⁷, R⁷SO₂C₀₋₆alkyl, SO₂R⁷, R⁷CON(R)C₀₋₆alkyl, NR⁸SO₂R⁷, COR⁷, COOR⁸, OSO₂R⁸, (R⁸)₂NCOC₀₋₆alkyl, SO₂N(R⁸)₂, N(R⁸)CON(R⁸)₂, NO₂, C₃₋₆cycloalkyl, C₃₋₆heterocycloalkyl or oxo;

n is 0, 1, 2, 3, 4 or 5;

B is O, N(R⁶)₂, or B is NR⁶ within a C₅₋₁₁heteroaryl wherein R⁶ forms a ring with Q;

X is O, CH₂, CO, S, SO, SO₂ or NR¹²;

R² is hydrogen, hydroxyl, halogen, C₁₋₁₀alkyl, C₂₋₁₀alkenyl, C₂₋₁₀alkynyl, C₁₋₁₀alkoxy, N(R¹⁰)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, CN, SR⁷, SO₂R⁸, SOR⁷, N(R⁸)COR⁷, N(R⁸)SO₂R⁷, COR⁷, COOR⁷, OSO₂R⁷, CON(R⁸)₂ or SO₂N(R⁸)₂;

R³ is hydrogen, C₁₋₁₀alkyl, C₂₋₁₀alkenyl, C₂₋₁₀alkynyl, C₆₋₁₀arylC₀₋₆alkyl, C₅₋₆heteroarylC₀₋₆alkyl, C₁₋₆haloalkyl or R⁷OC₁₋₆alkyl;

R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy; or

R³ and R⁴ form together a C₃₋₇heterocycloalkyl, and which may be substituted by one or more groups selected independently from hydrogen, halogen, C₁₋₆alkyl, C₁₋₆haloalkyl, COR¹¹, SO₂R¹¹, OR¹¹, cyano, oxo and SO₂N(R¹⁰)₂;

R⁵ is hydrogen, C₁₋₆alkyl, C₁₋₆alkoxy, C₁₋₆haloalkoxy or C₁₋₆haloalkyl; or

R⁴ and R⁵ form together a C₃₋₇heterocycloalkyl or a C₃₋₇cycloalkyl, and which may be substituted by one or more groups selected independently from hydrogen, halogen, C₁₋₆alkyl, C₁₋₆haloalkyl, COR¹¹, SO₂R¹¹, OR¹¹, cyano, oxo and SO₂N(R¹⁰)₂;

R⁶ is hydrogen, C₁₋₆alkyl, C₃₋₆cycloakylC₀₋₆alkyl, R⁷OC₁₋₆alkyl, C₁₋₆haloalkyl, C₁₋₆cyanoalkyl, (R¹⁰)₂NCOC₀₋₆alkyl or R¹¹SO₂C₁₋₆alkyl;

R⁷ is C₁₋₆alkyl, C₆₋₁₀arylC₀₋₆alkyl, C₅₋₆heteroarylC₀₋₆alkyl, C₃₋₇cycloalkylC₀₋₆alkyl or C₁₋₆haloalkyl;

R⁸ is hydrogen, C₁₋₆alkyl, C₁₋₆haloalkyl, C₃₋₇cycloalkylC₀₋₆alkyl, C₆₋₁₀arylC₀₋₆alkyl or C₅₋₆heteroarylC₀₋₆alkyl; or

R⁷ and R⁸ form together a C₅₋₆heteroaryl or C₃₋₇heterocycloalkyl;

whereby any aryl and heteroaryl under R¹, R³, R⁷ and R⁸ may be substituted by one or more groups selected independently from hydrogen, halogen, hydroxyl, C₁₋₆haloalkyl, CN, OR¹⁶, C₁₋₆alkyl, oxo, SR¹⁰, CON(R¹⁰)₂, N(R¹⁰)COR¹¹, SO₂R¹¹, SOR¹¹, N(R¹⁰)₂ and COR¹¹;

R⁹ is hydrogen, hydroxyl, halogen, C₁₋₆alkyl, C₁₋₆alkoxyC₀₋₃alkyl, C₁₋₆haloalkyl, COR¹¹, CON(R¹⁰)₂, N(R¹⁰)COR¹¹, SR¹⁰SOR¹¹, CN or SO₂R¹¹;

R¹⁰ is hydrogen, C₁₋₆alkyl or C₁₋₆haloalkyl;

R¹¹ is C₁₋₆alkyl or C₁₋₆haloalkyl; or

R¹⁰ and R¹¹ form together a C₃₋₇heterocycloalkyl, which may be substituted by one or more groups selected independently from hydrogen, halogen, hydroxyl, C₁₋₃alkyl, C₁₋₃alkoxy and cyano; and

R¹² is hydrogen, C₁₋₆alkyl, C₁₋₆haloalkyl, COR¹¹ or SO₂R¹¹;

or salts, solvates or solvated salts thereof.

In another embodiment of the invention there is provided compounds of formula I, wherein:

Q is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl or C₁₋₁₀alkyl;

R¹ is hydrogen, hydroxyl, halogen, C₁₋₁₀alkyl, C₂₋₁₀alkenyl, C₂₋₁₀alkynyl, C₁₋₁₀alkoxy, N(R¹⁰)₂, C₆₋₁₀arylC₀₋₆alkyl, C₁₋₆heteroarylCₒ₋₆alkyl, C₁₋₆haloalkyl, C₁₋₆haloalkylO, R⁸OC₀₋₆alkyl, CN, SR⁷, R⁷SO₂C₀₋₆alkyl, SO₂R⁷, R⁷CON(R⁸)C₀₋₆alkyl, NR⁸SO₂R⁷, COR⁷, COOR⁸, OSO₂R⁸, (R⁸)₂NCOC₀₋₆alkyl, SO₂N(R⁸)₂, N(R⁸)CON(R⁸)₂, NO₂, C₃₋₆cycloalkyl, C₃₋₆heterocycloalkyl or oxo;

n is 0, 1 or 2;

B is O or N(R⁶)₂;

X is O or CH₂;

R² is hydrogen, hydroxyl, halogen, C₁₋₁₀alkyl, C₁₋₁₀alkoxy, C₆₋₁₀arylC₀₋₆alkyl, C₅₋₆heteroarylC₀₋₆alkyl or C₁₋₆haloalkyl;

R³ is hydrogen, C₁₋₁₀alkyl, C₆₋₁₀arylC₀₋₆alkyl, C₅₋₆heteroarylC₀₋₆alkyl, C₁₋₆haloalkyl or R⁷OC₁₋₆alkyl;

R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy; or

R³ and R⁴ form together a C₃₋₇heterocycloalkyl, and which may be substituted by one or more groups selected independently from hydrogen, halogen, C₁₋₆alkyl, C₁₋₆haloalkyl, COR¹¹, SO₂R¹¹, OR¹¹, cyano, oxo and SO₂N(R¹⁰)₂;

R⁵ is hydrogen;

R⁶ is hydrogen, C₁₋₆alkyl, C₃₋₆cycloakylC₀₋₆alkyl, R⁷OC₁₋₆alkyl, C₁₋₆haloalkyl or C₁₋₆cyanoalkyl;

R⁷ is C₁₋₆alkyl, C₆₋₁₀arylC₀₋₆alkyl, C₅₋₆heteroarylC₀₋₆alkyl, C₃₋₇cycloalkylC₀₋₆alkyl or C₁₋₆haloalkyl;

R⁸ is hydrogen, C₁₋₆alkyl, C₁₋₆haloalkyl, C₃₋₇cycloalkylC₀₋₆alkyl, C₆₋₁₀arylC₀₋₆alkyl or C₅₋₆heteroarylC₀₋₆alkyl;

whereby any aryl and heteroaryl under R¹ may be substituted by one or more groups selected independently from hydrogen, halogen, hydroxyl, C₁₋₆haloalkyl, CN, OR¹⁰, C₁₋₆alkyl, oxo, SR¹⁰, CON(R¹⁰)₂, N(R¹⁰)COR¹¹, SO₂R¹¹, SOR¹¹, N(R¹⁰)₂ and COR¹¹; and

R¹⁰ is hydrogen, C₁₋₆alkyl or C₁₋₆haloalkyl;

or salts, solvates or solvated salts thereof.

In one embodiment of the invention Q is C₆₋₁₀arylC₀₋₄alkyl or C₅₋₁₁heteroarylC₀₋₄alkyl.

In another embodiment of the invention R¹ is hydrogen, halogen, C₁₋₄alkyl, C₁₋₄alkoxy, C₆₋₁₀arylC₀₄alkyl, C₅₋₆heteroarylC₀₋₄alkyl, C₁₋₄haloalkyl, COR⁷, R⁸OC₀₋₄alkyl, SO₂R⁷ or R⁷CON(R⁸)C₀₋₄alkyl.

In a further embodiment of the invention B is N(R⁶)₂, and R⁶ is hydrogen or C₁₋₃alkyl.

In one embodiment of the invention R² is hydrogen, halogen or C₁₋₄haloalkyl.

In yet another embodiment of the invention R³ is hydrogen, C₁₋₃alkyl or C₁₋₄haloalkyl.

In yet a further embodiment of the invention R³ and R⁴ form together a C₃₋₆heterocycloalkyl.

In another embodiment of the invention there is provided compounds of formula I, wherein;

Q is C₆₋₁₀arylC₀₋₄alkyl or C₅₋₁₁heteroarylC₀₋₄alkyl;

R¹ is hydrogen, halogen, C₁₋₄alkyl, C₁₋₄alkoxy, C₆₋₁₀arylC₀₋₄alkyl, C₅₋₆heteroarylC₀₋₄alkyl, C₁₋₄haloalkyl, COR⁷, R⁸OC₀₋₄alkyl, SO₂R⁷ or R⁷CON(R⁸)C₀₋₄alkyl;

n is 0, 1 or 2;

B is N(R⁶)₂;

X is O or C₁alkyl;

R² is hydrogen, halogen or C₁₋₄haloalkyl;

R³ is hydrogen, C₁₋₄alkyl or C₁₋₄haloalkyl;

R⁴ is hydrogen; or

R³ and R⁴ form together a C₃₋₇heterocycloalkyl;

R⁵ is hydrogen;

R⁶ is hydrogen;

R⁷ is C₁₋₄alkyl, C₆₋₁₀arylC₀₋₄alkyl or C₁₋₄haloalkyl;

R⁸ is C₃₋₇cycloalkylC₀₋₄alkyl;

whereby any aryl and heteroaryl under R¹, may be substituted by one or more groups selected independently from halogen, C₁₋₄haloalkyl and OR¹⁰; and

R¹⁰ is hydrogen or C₁₋₄haloalkyl;

or salts, solvates or solvated salts thereof.

In a further embodiment Q is phenyl, naftyl, benzothienyl, thiazole, pyrrolyl, pyridinyl, benzofuranyl, quinolinyl, phenylmethyl, tetralinyl, imidazothiazole or thienyl.

In one embodiment of the invention Q is substituted with 0, 1, 2, 3, 4 or 5 groups R¹, wherein the number of R¹ substituents on Q is designated by the term n. In another embodiment of the invention n is 0, 1 or 2.

In yet another embodiment Q is phenyl or naftyl substituted by one or more R¹.

In another embodiment R¹ is a halogen such as chloro, bromo, iodo and fluoro.

In yet a further embodiment R¹ is methyl, ethyl, propyl, butyl, pentyl, phenyl or naftyl.

In one embodiment R¹ is fluoromethyl, difluoromethyl, trifluoromethyl, fluoroethyl, difluoroethyl, bromomethyl or chloromethyl.

In another embodiment R¹ is methyl, trifluoromethyl or phenyl.

Alternatively R¹ is COR⁷, and R⁷ is a C₆₋₁₀aryl. In one embodiment R⁷ is phenyl.

In yet a further embodiment R¹ is methoxy, ethoxy, propoxy or fluoromethoxy, difluoromethoxy, trifluoromethoxy, fluoroethoxy or difluoroethoxy. In another embodiment R¹ is methoxy or fluoromethoxy.

In a further embodiment R¹ is hydrogen, NCOCH₃, pyridinyl, benzoyl or phenylSO₂.

In one embodiment B is N(R⁶)₂, and R⁶ is hydrogen or C₁₋₃alkyl. In another embodiment B is amine.

In yet another embodiment X is O or CH₂.

In a further embodiment R² is hydrogen, fluoromethyl, difluoromethyl, trifluoromethyl, fluoroethyl, difluoroethyl, bromomethyl or chloromethyl. In one embodiment R² is hydrogen or trifluoromethyl. In another embodiment R² is a halogen such as chloro, bromo, iodo and fluoro. In one embodiment R² is chloro.

In another embodiment R³ is hydrogen, methyl, ethyl, propyl, butyl or pentyl. In a further embodiment R³ is hydrogen, methyl or i-propyl.

In yet another embodiment R³ is fluoromethyl, difluoromethyl, trifluoromethyl, fluoroethyl, difluoroethyl, bromomethyl or chloromethyl. In one embodiment R³ is fluoroethyl.

In another embodiment R⁴ is hydrogen.

In a further embodiment R³ and R⁴ form together a C₃₋₆heterocycloalkyl. In yet a further embodiment R³ and R⁴ form together pyrrolidin.

In one embodiment R⁵ and R⁶ are hydrogen.

In another embodiment R⁷ is methyl or phenyl.

In yet another embodiment R⁸ is hydrogen or trifluoromethyl.

A further embodiment of the invention relates to compounds selected from the group consisting of:

3-bromo-N-(4-methyl-2,3,4,5-tetrahydro-1,4-benzoxazepin-7-yl)benzenesulfonamide;

N-(4-methyl-2,3,4,5-tetrahydro-1,4-benzoxazepin-7-yl)benzenesulfonamide;

2-chloro-N-(4-methyl-2,3,4,5-tetrahydro-1,4-benzoxazepin-7-yl)benzenesulfonamide;

3,5-dichloro-N-(4-methyl-2,3,4,5-tetrahydro-1,4-benzoxazepin-7-yl)benzenesulfonamide;

4-chloro-N-(4-methyl-2,3,4,5-tetrahydro-1,4-benzoxazepin-7-yl)benzenesulfonamide;

2,3-dichloro-N-(4-methyl-2,3,4,5-tetrahydro-1,4-benzoxazepin-7-yl)benzenesulfonamide;

3-methoxy-N-(4-methyl-2,3,4,5-tetrahydro-1,4-benzoxazepin-7-yl)benzenesulfonamide;

3-bromo-N-(9-chloro-4-isopropyl-2,3,4,5-tetrahydro-1,4-benzoxazepin-7-yl)benzenesulfonamide;

2,3-dichloro-N-(2,3,4,5-tetrahydro-1,4-benzoxazepin-7-yl)benzenesulfonamide;

4-chloro-N-(2,3,4,5-tetrahydro-1,4-benzoxazepin-7-yl)naphthalene-1-sulfonamide;

4-fluoro-N-(2,3,4,5-tetrahydro-1,4-benzoxazepin-7-yl)naphthalene-1-sulfonamide;

3-chloro-2-fluoro-N-(2,3,4,5-tetrahydro-1,4-benzoxazepin-7-yl)benzenesulfonamide;

5-chloro-N-(2,3,4,5-tetrahydro-1,4-benzoxazepin-7-yl)naphthalene-2-sulfonamide;

5-chloro-N-(2,3,4,5-tetrahydro-1,4-benzoxazepin-7-yl)naphthalene-1-sulfonamide;

N-(2,3,4,5-tetrahydro-1,4-benzoxazepin-7-yl)biphenyl-2-sulfonamide;

N-(2,3,4,5-tetrahydro-1,4-benzoxazepin-7-yl)biphenyl-3-sulfonamide;

N-(2,3,4,5-tetrahydro-1,4-benzoxazepin-7-yl)-2,3-dihydro-1-benzofuran-5-sulfonamide;

N-(2,3,4,5-tetrahydro-1,4-benzoxazepin-7-yl)quinoline-8-sulfonamide;

1-(3-chlorophenyl)-N-(2,3,4,5-tetrahydro-1,4-benzoxazepin-7-yl)methanesulfonamide;

N-(2,3,4,5-tetrahydro-1,4-benzoxazepin-7-yl)-5,6,7,8-tetrahydronaphthalene-2-sulfonamide;

N-(2,3,4,5-tetrahydro-1,4-benzoxazepin-7-yl)-5,6,7,8-tetrahydronaphthalene-1-sulfonamide;

2,3-dichloro-N-[4-(2-fluoroethyl)-2,3,4,5-tetrahydro-1,4-benzoxazepin-7-yl]benzenesulfonamide;

4-chloro-N-[4-(2-fluoroethyl)-2,3,4,5-tetrahydro-1,4-benzoxazepin-7-yl]naphthalene-1-sulfonamide;

2,3-dichloro-N-[(11aS)-2,3,11,11a-tetrahydro-1H,5H-pyrrolo[2,1-c][1,4]benzoxazepin-7-yl]benzenesulfonamide;

N-[(11aS)-2,3,11,11a-tetrahydro-1H,5H-pyrrolo[2,1-c][1,4]benzoxazepin-7-yl]naphthalene-1-sulfonamide;

N-[(11aS)-2,3,11,11a-tetrahydro-1H,5H-pyrrolo[2,1-c][1,4]benzoxazepin-7-yl]biphenyl-4-sulfonamide;

N-[(11aS)-2,3,11,11a-tetrahydro-1H,5H-pyrrolo[2,1-c][1,4]benzoxazepin-7-yl]-3-(trifluoromethyl)benzenesulfonamide;

3-bromo-N-[9-(trifluoromethyl)-2,3,4,5-tetrahydro-1,4-benzoxazepin-7-yl]benzenesulfonamide;

5-chloro-3-methyl-N-[9-(trifluoromethyl)-2,3,4,5-tetrahydro-1,4-benzoxazepin-7-yl]-1-benzothiophene-2-sulfonamide;

N-[4-methyl-5-({[9-(trifluoromethyl)-2,3,4,5-tetrahydro-1,4-benzoxazepin-7-yl]amino}sulfonyl)-1,3-thiazol-2-yl]acetamide;

2,3-dichloro-N-[9-(trifluoromethyl)-2,3,4,5-tetrahydro-1,4-benzoxazepin-7-yl]benzenesulfonamide;

3-(trifluoromethyl)-N-[9-(trifluoromethyl)-2,3,4,5-tetrahydro-1,4-benzoxazepin-7-yl]benzenesulfonamide;

N-[9-(trifluoromethyl)-2,3,4,5-tetrahydro-1,4-benzoxazepin-7-yl]naphthalene-1-sulfonamide;

2,3-dichloro-N-(2,3,4,5-tetrahydro-1H-2-benzazepin-8-yl)benzenesulfonamide;

4-chloro-N-(2,3,4,5-tetrahydro-1H-2-benzazepin-8-yl)naphthalene-1-sulfonamide;

N-(2,3,4,5-tetrahydro-1H-2-benzazepin-8-yl)biphenyl-2-sulfonamide;

N-(2,3,4,5-tetrahydro-1H-2-benzazepin-8-yl)quinoline-8-sulfonamide;

1-[3-chloro-5-(trifluoromethyl)pyridin-2-yl]-N-(2,3,4,5-tetrahydro-1H-2-benzazepin-8-yl)-1H-pyrrole-2-sulfonamide;

4-phenyl-N-(2,3,4,5-tetrahydro-1H-2-benzazepin-8-yl)-5-(trifluoromethyl)thiophene-3-sulfonamide;

4′-chloro-N-(2,3,4,5-tetrahydro-1H-2-benzazepin-8-yl)biphenyl-2-sulfonamide;

4-(phenylsulfonyl)-N-(2,3,4,5-tetrahydro-1H-2-benzazepin-8-yl)thiophene-2-sulfonamide;

5-chloro-3-methyl-N-(2,3,4,5-tetrahydro-1H-2-benzazepin-8-yl)-1-benzothiophene-2-sulfonamide;

2-bromo-N-(2,3,4,5-tetrahydro-1H-2-benzazepin-8-yl)benzenesulfonamide;

N-(2,3,4,5-tetrahydro-1H-2-benzazepin-8-yl)-2-(trifluoromethyl)benzenesulfonamide;

2-iodo-N-(2,3,4,5-tetrahydro-1H-2-benzazepin-8-yl)benzenesulfonamide;

2,6-dichloro-N-(2,3,4,5-tetrahydro-1H-2-benzazepin-8-yl)benzenesulfonamide;

N-(2,3,4,5-tetrahydro-1H-2-benzazepin-8-yl)-2-(trifluoromethoxy)benzenesulfonamide;

3,4-dichloro-N-(2,3,4,5-tetrahydro-1H-2-benzazepin-8-yl)benzenesulfonamide;

N-(2,3,4,5-tetrahydro-1H-2-benzazepin-8-yl)biphenyl-4-sulfonamide;

2,3-dichloro-N-(2-methyl-2,3,4,5-tetrahydro-1H-2-benzazepin-8-yl)benzenesulfonamide;

4-chloro-N-(2-methyl-2,3,4,5-tetrahydro-1H-2-benzazepin-8-yl)naphthalene-1-sulfonamide;

2-benzoyl-4-chloro-N-(2,3,4,5-tetrahydro-1H-2-benzazepin-8-yl)benzenesulfonamide; and

6-chloro-N-(2,3,4,5-tetrahydro-1H-2-benzazepin-8-yl)imidazo[2,1-b][1,3]thiazole-5- or salts, solvates or solvated salts thereof.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, neo-pentyl, n-hexyl, i-hexyl, etc. The term C₁₋₁₀alkyl having 1 to 10 carbon atoms and may be but are not limited to methyl, ethyl, n-propyl, i-propyl, n-butyl, i-butyl, s-butyl, tert-butyl, n-pentyl, i-pentyl, neo-pentyl, etc.

The term ‘C₀’ means a bond or does not exist. For example when “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂-₁₀alkenyl” having 2 to 10 carbon atoms and one or two double bonds, may be, but is not limited to vinyl, allyl, propenyl, butenyl, crotyl, pentenyl, hexenyl, heptenyl, octenyl, nanenyl, decenyl, and a butenyl group may for example be buten-2-yl, buten-3-yl or buten-4-yl.

In this specification, unless stated otherwise, the term “alkynyl” includes both straight and is branched chain alkynyl groups. The term “C₂-₁₀alkynyl” having 2 to 10 carbon atoms and one or two trippel bonds, may be, but is not limited to etynyl, propargyl, pentynyl, hexynyl, heptynyl, octynyl, nanynyl, decynyl and a butynyl group may for example be butyn-3-yl or butyn-4-yl.

The term “alkoxy”, unless stated otherwise, refers to radicals of the general formula —O—R, wherein R is selected from a hydrocarbon radical. The term “C₁-₁₀alkoxy” may include, but is not limited to methoxy, ethoxy, propoxy, isopropoxy, butoxy, t-butoxy, isobutoxy, cyclopropylmethoxy, allyloxy, propargyloxy, pentoxy, isopentoxy, etc.

In this specification, unless stated otherwise, the term “amine” or “amino” refers to radicals of the general formula —NRR′, wherein R and R′ are selected independently from hydrogen or a hydrocarbon radical. The term ‘N(R⁶)’ refers to a group wherein R⁶ may the same or different.

In this specification, unless stated otherwise, the term “cycloalkyl” refers to an optionally substituted, partially or completely saturated cyclic hydrocarbon ring system. The term “C₃₋₇cycloalkyl” may be, but is not limited to cyclopropyl, cyclobutyl, cyclopentyl, cyclohexyl, cycloheptyl or cyclopentenyl.

The term “heterocycloalkyl” denotes a non-aromatic, partially or completely saturated hydrocarbon group, which contains one ring and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon ring system with at least one unsaturated aromatic ring. Examples of “aryl” may be, but are not limited to phenyl, naphthyl or tetralinyl.

In this specification, unless stated otherwise, the term “heteroaryl” refers to an optionally substituted monocyclic or bicyclic hydrocarbon ring system with at least one unsaturated ring and containing at least one heteroatom selected independently from N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyrirlazinyl, pyrimidinyl, pyrazinyl, tetrazolyl, triazolyl, quinazolinyl or isoxazolyl. For the avoidance of doubt, a C₅heteroaryl refers to a 5 membered aromatic ring system containing at least one heteroatom.

In this specification, unless stated otherwise, the terms “arylalkyl” and “heteroarylalkyl” refer to a substituent that is attached via the alkyl group to an aryl or heteroaryl group.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl, bromopropyl or chloromethyl, etc. The term “C₁₋₆haloalkylO” may include, but is not limited to fluoromethoxy, difluoromethoxy, trifluoromethoxy, fluoroethoxy or difluoroethoxy.

The present invention relates to the compounds of formula I as hereinbefore defined as well as to the salts, solvates or solvated salts thereof. Salts for use in pharmaceutical compositions will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base. Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

Methods of Preparation

Detailed Process Description

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The specific sequence of reactions depicted under “General procedure” is not critical. For many of the compounds described the order of the reaction steps may be varied. The reactions were run until determined complete by LC-UV, LC-MS, TLC or NMR.

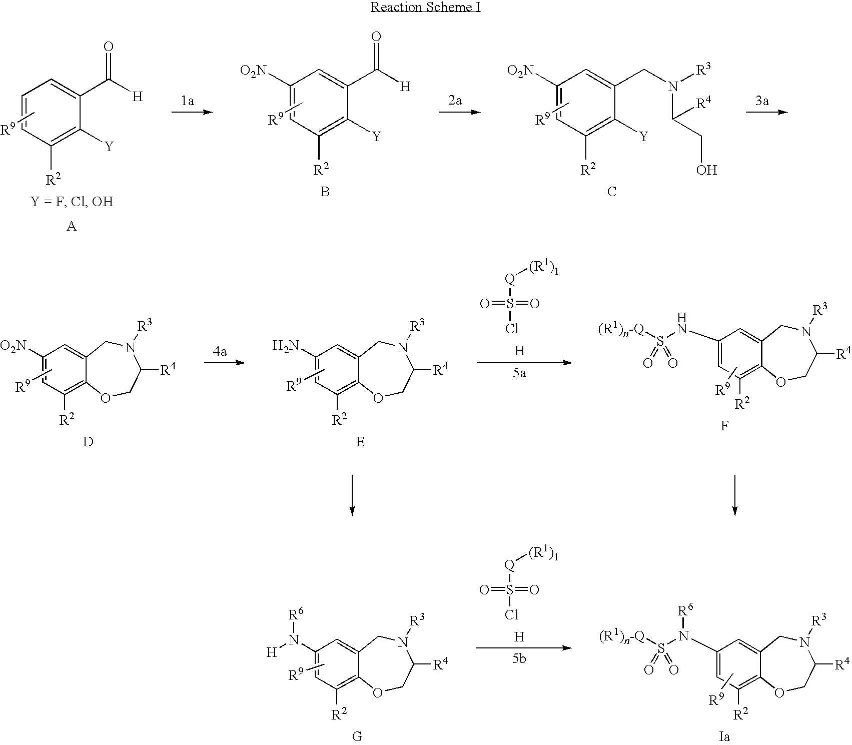

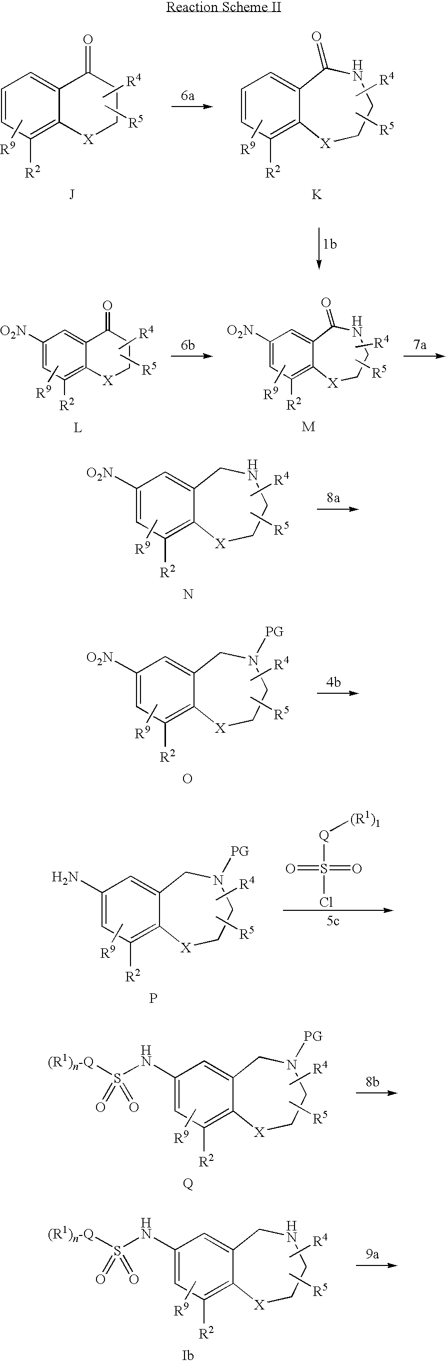

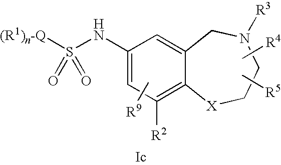

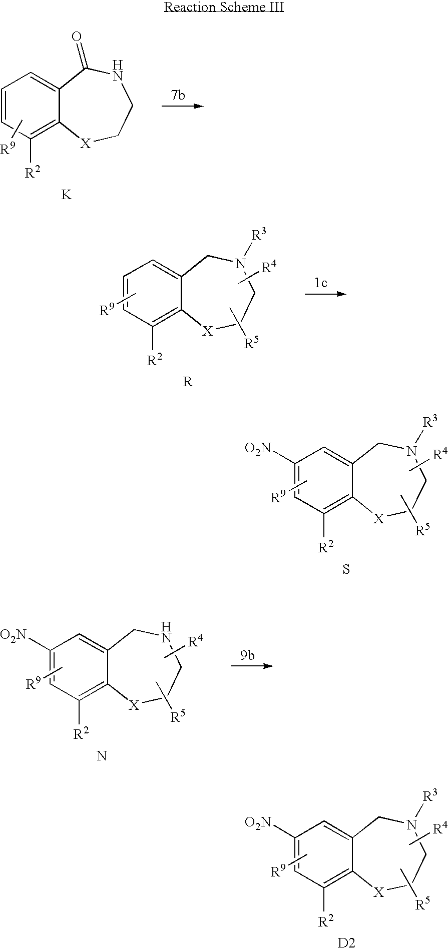

Step 1

A compound B may be prepared from a compound A via nitration (1c). The nitration may be performed using for example sodium nitrite or potassium nitrite in a solvent such as trifluoroacetic acid or sulfuric acid at temperatures between 0 and 60° C., preferably at temperatures between 0° C. and room temperature for reaction times between 1 and 10 hours. The nitration may also be performed using nitric acid in a solvent such as TFA or sulfuric acid at temperatures between −10° C. and RT for reaction times between 1 and 10 h. The product may be isolated by extraction, precipitation or column chromatography. The same method can be used to transform a compound R into a compound S (1c) or a compound K into a compound M (1b).

Step 2

A compound C may be prepared from a compound B using reductive amination (2a). Typically B may be mixed with a carbonyl compound such as an aldehyde or a ketone in the presence of a reducing agent such as sodium borohydride, sodium cyanoborohydride, sodium triacetoxyborohydride or hydrogen in the presence of a suitable catalyst such as for example described in “Advanced Organic Chemistry, Reactions, Mechanisms and Structure”, J. March, John Wiley & Sons, New York, 1992. An acid such as formic acid or acetic acid may be added to control the pH of the reaction. The reaction may be performed in a solvent such as water, methanol, ethanol, dichloromethane, THF, formic acid, acetic acid or mixtures thereof at temperatures between 0° and the reflux temperature of the solvent, preferably at RT. The reaction mixture may be either worked up by extraction and then purified by column chromatography or the reaction mixture may be concentrated and purified by column chromatography.

Step 3

A compound C may be transformed into a compound D (3c) via intramolecular aromatic nucleophilic substitution where Y═F or Cl. Typically compound C is dissolved in a solvent such as THE, dioxane or DMF and a base such as sodium hydride or sodium methoxide is added. The reaction may be performed at temperatures between RT and the reflux temperature of the solvent for reaction times between 1 and 24 h. The product may be isolated by extraction, precipitation or column chromatography.

Alternatively, when Y═OH, an intramolecular ringclosure of Mitsunobo type may be used. Typically a compound C may be dissolved in a solvent such as DMF, THF or dichloromethane or mixtures thereof. A phosphine compound such as triphenylphosphine or tributylphosphine and an activating agent such as diethyl azodicarboxylate or diisopropyl azodicarboxylate are added, preferably at temperatures between −10° C. and ambient temperature. The reaction may be performed at temperatures between −15° C. and the reflux temperature of the solvent, preferably at ambient temperature for reaction times between 1 and 24 h. The product may be isolated by extraction, precipitation or column chromatography.

Step 4

The reduction of a compound D to a compound E (4a) may be performed using hydrogenation with a suitable catalyst such as palladium on charcoal in a solvent such as methanol, ethanol, EtOAc, acetic acid or mixtures thereof, optionally in the presence of for example hydrochloric acid or ammonia. For other suitable catalysts see for example “Comprehensive Organic Transformations, a Guide to Functional Group Preparation”, R C. Larock, John Wiley & sons, New York, 1999. Hydrogen sources may be hydrogen gas at atmospheric or increased pressure or for example ammonium formate. Other reducing agents that might be used are for example tin(II) chloride hydrate in solvents such as ethanol or EtOAc. The reaction might be performed at temperatures between RT and the reflux temperature of the solvent. The product may be isolated by solvent removal, extraction, precipitation or column chromatography.

The same method can be used to transform a compound O into a compound P (4b).

Step 5

A compound E may be transformed into a compound F (5a) by reaction with a compound H in a solvent such as DMF, N-methylpyrrolidin, acetonitrile, dioxane, chloroform or dichloromethane or mixtures thereof in the presence of a base such as pyridine, triethylamine, PS-DIEA or DIPEA at temperatures between 0° C. and the reflux temperature of the solvent. The product may be isolated by extraction, precipitation or column chromatography.

The same method can be used to transform a compound G into a compound Ia (5b) or a compound P into a compound Q (5c).

Step 6

A compound J may be transformed into compound K via the Schmidt rearrangement (6a). Compound J and sodium azide may be dissolved in a solvent such as benzene, TFA or acetic acid. Sulfuric acid may be added at temperatures below 5° C., typically between −10° C. and 5° C. The reaction may be performed at temperatures between RT and the reflux temperature of the solvent. The mixture may then be poured onto ice or water, the mixture may be made basic with a base such as ammonia, potassium carbonate or sodium hydroxide. The mixture may be stirred at RT for 1-20 h and the product may be isolated by extraction, precipitation or column chromatography.

The same method can be used to transform a compound L into a compound M (6b).

Step 7

The reduction of compound M to compound N (7c) may be performed with a reducing agent such as borane or lithium aluminum hydride in a solvent such as tetrahydrofuran or diethyl ether at temperatures between 0° and the reflux temperature of the solvent, preferably between 25° and the reflux temperature. The product may be isolated by column chromatography or by extraction.

The same method can be used to transform a compound K into a compound R (7b).

Step 8

A compound N may be transformed into a compound O (8a) using standard protecting group chemistry. The same methodology can be used to transform a compound Q into a compound Ib (8b).

Conventional procedures for using such protecting groups, as well as examples of suitable protecting groups are described in, for example, “Protective Groups in Organic Synthesis” T. W. Green, P. G. M. Wuts, Wiley-Interscience, New York, 1999.

Step 9

A compound Ic may be prepared from a compound Ib (9a) by alkylation with a compound R³Y² where Y² may be a suitable leaving group such as a halogen, mesylate or triflate, such as for example described in “Comprehensive Organic Transformations, a Guide to Functional Group Preparation”, R. C. Larock, John Wiley & sons, New York, 1999. Typically, Ib and R³Y² are mixed in a solvent such as DMF, ethanol, dichloromethane or toluene in the presence of a base such as sodium bicarbonate, sodium carbonate, potassium carbonate, triethylamine or diisopropylethylamine and optionally, if Y═Cl, Br, a catalytic amount of potassium iodide. The reaction may be performed at temperatures between 25° and the reflux temperature of the solvent and the reaction time may be between 1 and 100 hours. The reaction mixture may be either worked up by extraction and then purified by column chromatography or the reaction mixture may be concentrated and purified by column chromatography. The reaction temperature may be elevated above the reflux temperature of the solvent and reaction times shortened by the use of microwave heating.

A compound Ic may also be prepared from a compound Ib by first preparing the amide or carbamate followed by reduction using an appropriate reducing agent. The amide may for example be prepared by reaction of Ib with an acid chloride or with a carboxylic acid in the presence of a coupling reagent, such as for example described in “Comprehensive Organic Transformations, a Guide to Functional Group Preparation”, R. C. Larock, John Wiley & sons, New York, 1999. The carbamate may be prepared by the reaction of an alkylchloroformate with a compound Ib in a solvent such as dichloromethane in the presence of a base such as triethylamine or pyridine at temperatures between 0° C. and the reflux temperature of the solvent. The reduction of the carbamate or the amide may be performed with a reducing agent such as lithium aluminum hydride in a solvent such as tetrahydrofuran or diethyl ether at temperatures between 0° and the reflux temperature of the solvent, preferably between 25° and the reflux temperature. The reduction of the amide may also be performed using borane as the reducing agent. The methods described under “step 2” can also be used for the transformation of compound Ib into compound Ic. The same methods can be used to transform a compound N into a compound D2.

Intermediates

One embodiment of the invention relates to intermediates of formula II

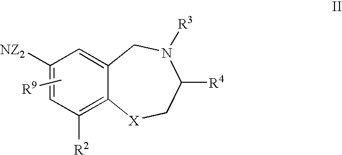

wherein Z is N or O, and wherein X, R², R³, R⁴ and R⁹ are defined as above, which may be used in the preparation of compounds of formula I.

Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream, for rectal administration, e.g. as a suppository, or for inhalation.

In general, the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

Medical Use

Interestingly, it has been found that the compounds according to the present invention are useful in therapy. The compounds of formula I, or salts, solvates or solvated salts thereof, as well as their corresponding active metabolites or prodrugs, exhibit a high degree of potency and selectivity for 5-hydroxy-tryptamine 6 (5-HT6) receptors. Accordingly, the compounds of the present invention are expected to be useful in the treatment of conditions associated with excessive activation of 5-HT6 receptors.

The compounds of formula I are expected to be suitable for the treatment of disorders relating to or affected by the 5-HT6 receptor including cognitive, personality, behaviour, psychiatric and neurodegenerative disorders.

Examples of such disorder may be selected from the group comprising of Alzheimer's disease anxiety, depression, convulsive disorders such as epilepsy, personality disorders, obsessive compulsive disorders, migraine, cognitive disorders such as memory dysfunction, sleep disorders, feeding disorders such as anorexia, obesity, bulimia, panic attacks, withdrawal from drug abuse, schizophrenia, cognitive impairment associated with schizophrenia, attention deficit hyperactive disorder (ADHD), attention deficit disorder (ADD), dementia, memory loss, disorders associated with spinal trauma and/or head injury, stroke, diabetes type 2, binge disorders, bipolar disorders, psychoses, Parkinson's disease, Huntington's disease, neurodegenerative disorders characterized by impaired neuronal growth, and pain.

Further relevant disorders may be selected from the group comprising gastro-intestinal disorders such as gastro-esophageal reflux disease (GERD) and irritable bowel syndrome (IBS).

The compounds may also be used for treatment of tolerance to 5-HT6 activators.

One embodiment of the invention relates to the compounds of formula I as hereinbefore defined, for use in therapy.

Another embodiment of the invention relates to the compounds of formula I as hereinbefore defined, for use in treatment of 5-HT6 mediated disorders.

A further embodiment of the invention relates to the compounds of formula I as hereinbefore defined, for use in treatment of Alzheimer's disease.

Another embodiment of the invention relates to the compounds of formula I as hereinbefore defined, for use in treatment of cognitive disorders such as for example cognitive impairment associated with schizophrenia.

Yet a further embodiment of the invention relates to the compounds of formula I as hereinbefore defined, for use in treatment of obesity.

One embodiment of the invention relates to the compounds of formula I as hereinbefore defined, for use in treatment of Parkinson's disease.

Another embodiment of the invention relates to the use of the compounds of formula I as hereinbefore defined, in the manufacture of a medicament for treatment of 5-HT6 mediated disorders, Alzheimer's disease, cognitive disorders, cognitive impairment associated with schizophrenia, obesity and/or Parkinson's disease, and any other disorder mentioned above.

A further embodiment of the invention relates to a method of treatment of 5-HT6 mediated disorders, Alzheimer's disease, cognitive disorders, cognitive impairment associated with schizophrenia, obesity and/or Parkinson's disease, and any other disorder mentioned above, comprising administering to a mammal, including man in need of such treatment, a therapeutically effective amount of the compounds of formula I, as hereinbefore defined.

Yet another embodiment of the invention relates to a pharmaceutical composition comprising a compound of formula I as hereinbefore defined, for use in treatment of 5-HT6 mediated disorders, Alzheimer's disease, cognitive disorders, cognitive impairment associated with schizophrenia, obesity and/or Parkinson's disease, and any other disorder mentioned above.

One embodiment of the invention relates to an agent for the treatment of 5-HT6 mediated disorders, Alzheimer's disease, cognitive disorders, cognitive impairment associated with schizophrenia, obesity and/or Parkinson's disease, and any other disorder mentioned above, which comprises as active ingredient a compound of formula I as hereinbefore defined.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the terms “inhibitor” and “antagonist” mean a compound that by any means, partly or completely, blocks the transduction pathway leading to the production of a response by the agonist.

The compounds according to the present invention are modulators of the 5-HT6 receptors, and may be inhibitors, as well as agonists, inverse-agonists or partial-agonist.

The term “disorder”, unless stated otherwise, means any condition and disease associated with 5-HT6 receptor activities.

Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of modulators of 5-HT6 related activity in laboratory animals such as cats, dogs, rabbits, monkeys, rats and mice, as part of the search for new therapeutics agents.

### EXAMPLES

General Methods

The invention will now be illustrated by the following Examples in which, generally:

operations were carried out at ambient or room temperature, i.e. in the range 17 to 25° C. and under an atmosphere of an inert gas such as argon unless otherwise stated. All solvents used were analytical grade and commercially available anhydrous solvents were used for reactions;

evaporations were carried out by rotary evaporation in vacuo and work-up procedures were carried out after removal of residual solids by filtration;

HPLC analyses were performed on an Agilent HP1000 system consisting of G1379A Micro Vacuum Degasser, G1312A Binary Pump, G1367A Wellplate auto-sampler, G1316A Thermostatted Column Compartment and G1315B Diode Array Detector. Column: X-Terra MS, Waters, 4.6×50 mm, 3.5 μm. The column temperature was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5% acetonitrile in MilliQ Water;

thin layer chromatography (TLC) was performed on Merck TLC-plates (Silica gel 60 F₂₅₄) and UV visualized the spots. Flash chromatography was preformed on a Combi Flash® Companion™ using RediSep™ normal-phase flash columns or on Merck Silica gel 60 (0.040-0.063 mm) Typical solvents used for flash chromatography were mixtures of chloroform/methanol, toluene/ethyl acetate and ethyl acetate/hexanes;

¹H and ¹³C NMR spectra were recorded at 400 MHz for proton and 100 MHz for carbon-13 either on a Varian Unity+400 NMR Spectrometer equipped with a 5 mm BBO probe with Z-gradients, or a Broker Avance 400 NMR spectrometer equipped with a 60 μl dual inverse flow probe with Z-gradients, or a Bruker DPX400 NMR spectrometer equipped with a 4-nucleus probe equipped with Z-gradients. The following reference signals were used: the middle line of DMSO-d₆ δ 2.50 (¹H); the middle line of CD₃OD δ 3.31 (¹H); acetone-d₆ 2.04 (¹H); and CDCl₃ δ 7.26 (¹H) (unless otherwise indicated);

mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN). Alternatively, mass spectra were recorded on a Waters LCMS system (Sample Manager 2777C, 1525μ binary pump, 1500 Column Oven, ZQ, PDA2996 and ELS detector, Sedex 85). Separation was performed using a Zorbax column (C8, 3.0×50 mm, 3 μm) supplied by Agilent Technologies. A four minutes linear gradient was used starting at 100% A (A: 95:5 10 mM NH₄OAc:MeOH) and ending at 100% B (MeOH). The ZQ was equipped with a combined APPI/APCI ion source and scanned in the positive mode between m/z 120-800 using a scan time of 0.3 s. The APPI repeller and the APCI corona were set to 0.86 kV and 0.80 μA, respectively. In addition, the desolvation temperature (300° C.), desolvation gas (400 L/Hr) and cone gas (5 L/Hr) were constant for both APCI and APPI mode;

preparative chromatography was run on a Gilson auto-preparative HPLC with a diode array detector. Column:)(Terra MS C8, 19×300 mm, 7 μm. Gradients with MeCN and (95:5 0.1M NH₄OAc:MeCN) were used. Flow rate: 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Gradients with MeCN and (95:5 0.1M NH₄OAc:MeCN) were used. Flow rate: 10 ml/min;

GC-MS analysis was performed on a GC-MS (GC 6890, 5973N MSD) supplied by Agilent Technologies. The column used was a DB-5 MS, ID 0.25 mm×30 m, 0.25 μm. A linear temperature gradient was applied starting at 40° C. (hold 1 min) and ending at 300° C. (hold 1 min), 25° C./minute. The MS was equipped with a CI ion source and the reactant gas was methane. The MS was scanned between m/z 50-500 and the scan speed was set to 3.25 scan/s. Alternatively mass spectra (EI-DI) were recorded on a Finigan MAT SSQ 710 spectrometer;

microwave heating was performed in a Creator, Initiator or Smith Synthesizer Single-mode microwave cavity producing continuous irradiation at 2450 MHz;

yields, where present, are not necessarily the maximum attainable;

intermediates were not necessarily fully purified but their structures and purity were assessed by thin layer chromatographic, HPLC, infra-red (IR), MS and/or NMR analysis;

the following abbreviations have been used:

HPLC high performance liquid chromatography

LC liquid chromatography

MS mass spectometry

TFA trifluroacetic acid

THF tetrahydrofuran

DMF dimethyformamide

DIPEA N,N-diisopropylethylamine

DMSO dimethylsulfoxide

NMP 1-methyl-2-pyrrolidinone

MeOH methanol

RT room temperature

PS-DIEA Polystyrene-bound diethylamine

PG Protecting Group

PS Trisamine tris-(2-aminoethyl)-amine polystyrene

EtOAc ethyl acetate

The invention will now be illustrated by the following non-limiting examples.

Starting material 5,6,7,8-tetrahydronaphthalene-1-sulfonyl chloride and 5,6,7,8-tetrahydronaphthalene-2-sulfonyl chloride were prepared according to Bioorg. Med. Chem. Lett. (1998) 6, 869-876.

### Example 1

### (i): 3-Bromo-N-(4-methyl-2,3,4,5-tetrahydro-1,4-benzoxazepin-7-yl)benzenesulfonamide

4-Methyl-2,3,4,5-tetrahydro-1,4-benzoxazepin-7-amine (26 mg, 0.145 mmol) was dissolved in chloroform:acetonitrile (1:1, 1.5 ml) and was added to a solution of 3-bromobenzenesulfonyl chloride (44 mg, 0.174 mmol) and pyridine (23 μl, 0.29 mmol) in chloroform: acetonitrile (1:1, 1 ml). The mixture was stirred at ambient temperature for 30 min and methanol (1 ml) was added. The solvents were evaporated and the product was isolated by preparative HPLC to give a dry film (43 mg, 76%). ¹H NMR (400 MHz, DMSO-4) δ ppm 7.68-7.79 (2H, m) 7.44 (2H, t) 6.72-6.85 (3H, m) 3.83-3.89(2H, m) 3.48-3.54 (2H, m) 2.78-2.84 (2H, m) 2.19 (3H, s); MS ESI m/z M+H⁺ 397, 399; M−H⁺ 395, 397.

### (ii) 2-{[(2-Hydroxyethyl)(methyl)amino]methyl}-4-nitrophenol

2-(Methylamino)ethanol (3.9 ml, 48 mmol) and acetic acid (2.6 ml, 48 mmol) were dissolved in THF (85 ml). 2-Hydroxy-5-nitrobenzaldehyde (8.1 g, 48 mmol) was added and the mixture was cooled to 0° C. Sodium triacetoxyborohydride (15.4 g, 73 mmol) was added and the cooling bath was removed. The mixture was stirred at ambient temperature for 5 h. The pH was adjusted to 1 by the addition of hydrochloric acid (10%). The precipitate formed was removed by filtration. The filtrate was washed with dichloromethane, neutralized with sodium hydroxide (aq, 1M) and extracted (×5) with dichlormethane. The combined organic phases were dried (MgSO₄) and the solvents were evaporated. The product was isolated by column chromatography on silica eluting with chloroform:methanol (gradient from 18:1 to 12:1) to give the title compound (1.8 g, 17%). MS ESI m/z M+H⁺ 227; M−H⁺ 225.

### (iii) 4-Methyl-7-nitro-2,3,4,5-tetrahydro-1,4-benzoxazepine

2-{[(2-Hydroxyethyl)(methypamino]methyl}-4-nitrophenol (1.84 g, 8.14 mmol) and triphenylphosphine (3.20 g, 12.2 mmol) were dissolved in THF (50 ml) and dichloromethane (20 ml). The mixture was cooled to 0° C. and diethyl azodicarboxylate (2.15 ml, 12.2 mmol) was added. The mixture was stirred under argon atmosphere at ambient temperature for 4 h. Water was added and the mixture was extracted with EtOAc. The organic phase was dried (MgSO₄) and the solvent was evaporated. The residue was purified by flash chromatography on silica eluting with hexane:EtOAc (gradient 50-100% EtOAc) to give the title compound (1.02 g, 60%). MS ESI m/z M+H⁺ 209.

### (iv) 4-Methyl-2,3,4,5-tetrahydro-1,4-benzoxazepin-7-amine

4-Methyl-7-nitro-2,3,4,5-tetrahydro-1,4-benzoxazepine (1.02 g, 4.81 mmol) was dissolved in ethanol (35 ml) and 10% palladium on charcoal (100 mg) was added. The mixture was hydrogenated at 50 psi pressure of hydrogen gas for 1 h. The mixture was filtered through Al₂O₃ (neutral) and celite. The solvent was evaporated to give the title compound in quantitative yield. MS ESI m/z M−H⁺ 177.

### Example 2-7

Examples 2-7 were prepared according to the method presented in example 1(i):

| Example | | | MS (ESI) | |
|---|---|---|---|---|
| no | Name | 1H NMR (400 MHz) | m/z | yield |
| 2 | N-(4-methyl-2,3,4,5- | (DMSO-d6) δ | M + H+ | 54% |
| | tetrahydro-1,4-benzoxazepin- | ppm 7.68-7.72 (2H, | 319; M − H+ | |
| | 7-yl)benzenesulfonamide | m) | 317 | |
| | | 7.56-7.62 (1H, m) | | |
| | | 7.49-7.56 (2H, m) | | |
| | | 6.87 (1H, d) 6.83 (1H, | | |
| | | d) 6.79 (1H, d) | | |
| | | 3.85-3.89 (2H, | | |
| | | m) 3.51 (2H, s) | | |
| | | 2.79-2.84 (2H, | | |
| | | m) 2.18 (3H, s) | | |
| 3 | 2-chloro-N-(4-methyl- | (CHLOROFORM- | M + H+ | 22% |
| | 2,3,4,5-tetrahydro-1,4- | d) δ ppm 7.93 (1H, | 353; M − H+ | |
| | benzoxazepin-7- | dd) | 351 | |
| | yl)benzenesulfonamide | 7.42-7.53 (2H, m) | | |
| | | 7.28-7.34 (1H, m) | | |
| | | 6.88-6.95 (2H, m) | | |
| | | 6.81 (1H, d) | | |
| | | 3.98-4.04 (2H, m) | | |
| | | 3.65 (2H, s) | | |
| | | 2.96-3.01 (2H, m) | | |
| | | 2.35 (3H, s) | | |
| 4 | 3,5-dichloro-N-(4-methyl- | (CHLOROFORM- | M + H+ | 19% |
| | 2,3,4,5-tetrahydro-1,4- | d) δ ppm | 387, 389; | |
| | benzoxazepin-7- | 7.47-7.56 (3H, m) | M − H+ | |
| | yl)benzenesulfonamide | 6.83-6.93 (2H, m) | 385, 387 | |
| | | 6.62 (1H, br. s.) | | |
| | | 4.09-4.16 (2H, | | |
| | | m) 3.66 (2H, s) | | |
| | | 3.05-3.11 (2H, | | |
| | | m) 2.48 (3H, s) | | |
| 5 | 4-chloro-N-(4-methyl- | (CHLOROFORM- | M + H+ | 33% |
| | 2,3,4,5-tetrahydro-1,4- | d) δ ppm 7.59 (2H, | 353, 355; | |
| | benzoxazepin-7- | d) 7.34 (2H, d) | M − H+ | |
| | yl)benzenesulfonamide | 6.78-6.88 (3H, | 351, 353 | |
| | | m) 3.94-3.99 (2H, | | |
| | | m) 3.57 (2H, s) | | |
| | | 2.88-2.93 (2H, | | |
| | | m) 2.31 (3H, s) | | |
| 6 | 2,3-dichloro-N-(4-methyl- | (CHLOROFORM- | M + H+ | 13% |
| | 2,3,4,5-tetrahydro-1,4- | d) δ ppm | 387, 389, | |
| | benzoxazepin-7- | 7.86-7.91 (1H, m) | 391; M − H+ | |
| | yl)benzenesulfonamide | 7.61-7.67 (1H, m) | 385, | |
| | | 7.24-7.28 (1H, | 387, 389 | |
| | | m) 6.81-6.95 (3H, | | |
| | | m) | | |
| | | 3.96-4.04 (2H, m) 3.63 (2H, | | |
| | | s) 2.93-3.00 (2H, | | |
| | | m) 2.36 (3H, s) | | |
| 7 | 3-methoxy-N-(4-methyl- | (CHLOROFORM- | M + H+ | 16% |
| | 2,3,4,5-tetrahydro-1,4- | d) δ ppm | 349; M − H+ | |
| | benzoxazepin-7- | 7.23-7.34 (2H, m) | 347 | |
| | yl)benzenesulfonamide | 7.16 (1H, br. s.) | | |
| | | 7.00-7.06 (1H, m) | | |
| | | 6.85 (2H, br. s.) | | |
| | | 6.67 (1H, br. s.) | | |
| | | 4.04-4.10 (2H, m) | | |
| | | 3.75 (3H, s) 3.61 (2H, | | |
| | | s) 2.99-3.05 (2H, | | |
| | | m) 2.41 (3H, s) | | |

### Example 8

### (i): 3-Bromo-N-(9-chloro-4-isopropyl-2,3,4,5-tetrahydro-1,4-benzoxazepin-7-yl)benzenesulfonamide

The title compound was prepared according to the method in example 1(i) starting from 9-chloro-4-isopropyl-2,3,4,5-tetrahydro-1,4-benzoxazepin-7-amine to give the title compound (35 mg, 51%). ¹H NMR (400 MHz, CHLOROFORM-d) δ ppm 7.88 (1H, s) 7.65 (2H, m) 7.32 (1H, t) 7.01 (1H, d) 6.71 (1H, d) 4.13-4.18 (2H, m) 3.68 (2H, s) 3.10-3.15 (2H, m) 2.96-3.05 (1H, m) 1.08-1.12 (6H, m); MS ESI m/z M+H⁺ 459, 461; M−H⁺ 457, 459.

### (ii) 2-[(3-Chloro-2-fluorobenzyl)(isopropyl)amino]ethanol

2-(i-Propylamine)ethanol (1.83 ml, 15.8 mmol) and acetic acid (0.90 ml, 15.8 mmol) were dissolved in anhydrous THF (40 ml) and the mixture was cooled to 0° C. 3-Chloro-2-fluorobenzaldehyde (1.85 ml, 15.8 mmol) and sodium triacetoxyborohydride (5.0 g, 23.7 mmol) were added. The mixture was stirred at ambient temperature for 20 h. Saturated aqueous sodium hydrogen carbonate (8 ml) was added and the mixture was extracted with EtOAc. The organic phase was dried (MgSO₄) and the solvent was evaporated to give the title compound (3.9 g). MS ESI m/z M+H⁺ 246, 248.

### (iii) 9-Chloro-4-isopropyl-2,3,4,5-tetrahydro-1,4-benzoxazepine

2-[(3-Chloro-2-fluorobenzyl)(isopropyl)amino]ethanol (3.9 g, 15.8 mmol) was dissolved in THF:DMF (2:1, 100 ml) and the solution was added dropwise to a slurry of sodium hydride (0.80 g, 31.5 mmol) in THF:DMF (2:1, 75 ml). The reaction mixture was stirred at ambient temperature for 30 min and at 50° C. for 2.5 h. Methanol was added dropwise to quench the reaction. The mixture was neutralized with Dowex H⁺ resins and the resins were removed by filtration. The mixture was concentrated by evaporation. Water (50 ml) was added followed by aqueous sodium hydroxide (1M) until pH 10 was reached. The mixture was extracted with diethyl ether. The organic phase was dried (MgSO₄), evaporated and the residue was purified by preparative HPLC to give the title compound (0.64 g, 18% over 2 steps). MS ESI m/z M+H⁺ 226, 228.

### (iv) 9-Chloro-4-isopropyl-7-nitro-2,3,4,5-tetrahydro-1,4-benzoxazepine

9-Chloro-4-isopropyl-2,3,4,5-tetrahydro-1,4-benzoxazepine was dissolved in TFA (0.8 ml) and the mixture was cooled to 0° C. Nitric acid (0.34 ml) was added and the mixture was s stirred at 0° C. for 35 min. The mixture was poured onto ice and the solid was isolated by filtration and washed with water to give the title compound (64 mg, 53%). MS ESI m/z M+H⁺ 271, 273.

### (v) 9-Chloro-4-isopropyl-2,3,4,5-tetrahydro-1,4-benzoxazepin-7-amine

9-Chloro-4-isopropyl-7-nitro-2,3,4,5-tetrahydro-1,4-benzoxazepine (64 mg, 0.24 mmol) was suspended in ethanol (2.7 ml) and tin(II) chloride dihydrate (265 mg, 1.18 mmol) was added followed by hydrochloric acid (4 drops). The mixture was heated at 70° C. for 2 h and at 40° C. for 16 h. Ice was added followed by aqueous sodium hydrogen carbonate and the mixture was extracted with EtOAc (×3) and dichloromethane (×3). The combined organic phases were dried (MgSO₄) and the solvents were evaporated. The residue was purified by preparative HPLC to give the title compound (47 mg, 84%). ¹H NMR (400 MHz, METHANOL-d₄) δ ppm 6.68 (1H, d) 6.55 (1H, d) 4.04-4.10 (2H, m) 3.89 (2H, s) 3.15-3.27 (3H, m) 1.22 (6 H, d). MS ESI m/z M+H⁺ 241, 243.

### Example 9

### (i): 2,3-Dichloro-N-(2,3,4,5-tetrahydro-1,4-benzoxazepin-7-yl)benzenesulfonamide

4-(Trifluoroacetyl)-2,3,4,5-tetrahydro-1,4-benzoxazepin-7-amine (183 mg, 0.70 mmol) was dissolved in chloroform:acetonitrile (3:1, 5 ml) and pyridine (116 μl, 1.43 mmol) was added followed by 2,3-dichlorobenzenesulfonyl chloride (171 mg, 0.69 mmol). The mixture was stirred at ambient temperature for 1 h and methanol (1 ml) was added. The solvents were evaporated and the residue was dissolved in chloroform (7 ml) and aqueous sodium hydroxide (2 M, 7 ml) was added. The mixture was stirred at ambient temperature for 1 h and water (25 ml) was added. Concentrated hydrochloric acid was added until acidic pH was reached and then sodium hydrogen carbonate was added to basic pH was reached. The mixture was extracted with chloroform (×2). The organic phase was dried (MgSO₄) and the solvent was evaporated. The residue was purified by preparative HPLC to give the acetate of the title compound as a solid (209 mg, 69%). ¹H NMR (400 MHz, DMSO-d₆) δ ppm 7.85-8.02 (2H, m) 7.51 (1H, t) 6.76-6.90 (3H, m) 3.81-3.87 (2H, m) 3.65 (2H, s) 2.93-2.99 (2H, m); MS ESI m/z M+H⁺ 373, 375; M−H⁺ 373, 375.

### (ii) 3,4-Dihydro-1,4-benzoxazepin-5(2H)-one

4-Chromanone (25 g, 169 mmol) and sodium azide (33.2 g, 510 mmol) were dissolved in acetic acid (335 ml). The solution was cooled to 0° C. and concentrated sulfuric acid (50 ml) was added dropwise. The mixture was heated at reflux for 4 h and then cooled to RT. The to mixture was poured onto ice (500 ml) and concentrated ammonium hydroxide was added until basic pH was reached. The mixture was stirred at ambient temperature for 20 h and the solid formed was collected by filtration to give the title compound (15 g, 54%). MS ESI m/z M+H⁺ 164.

### (iii) 7-Nitro-3,4-dihydro-1,4-benzoxazepin-5(2H)-one

3,4-Dihydro-1,4-benzoxazepin-5(2H)-one (15 g, 92 mmol) was dissolved in concentrated sulfuric acid and the mixture was cooled to 0° C. Potassium nitrite (10.4 g, 103 mmol) was added portionwise. The mixture was stirred at 0° C. for 30 min and at room temperature for 4 h. The mixture was poured onto ice (1000 ml) and the solid formed was collected by filtration. The solid was suspended in boiling EtOAc and then cooled to RT. The solid was collected by filtration to give the title compound (8.21 g, 43%). MS ESI m/z M+H⁺ 209; M−H⁺ 207.

### (iv) 7-Nitro-2,3,4,5-tetrahydro-1,4-benzoxazepine

7-Nitro-3,4-dihydro-1,4-benzoxazepin-5(2H)-one (0.99 g, 4.7 mmol) was suspended in THF (7 ml) and BH₃ (1 M in THF, 19 ml, 19 mmol) was added. The mixture was heated at reflux for 4 h. The mixture was cooled to 0° C. and hydrochloric acid (4 M, 7 ml) was added. The mixture was heated at reflux for 1 h and then concentrated by evaporation. The residue was diluted with water (30 ml) and neutralized with solid sodium hydrogen carbonate. The mixture was extracted with EtOAc (3×). The organic phase was dried (MgSO₄) and the solvent was evaporated. The residue was purified by column chromatography on silica eluting with chloroform:methanol 10:1 containing 0.1% triethylamine to give the title compound as an oil (0.88 g, 96%). MS ESI m/z M+H⁺ 195.

### (v) 7-Nitro-4-(trifluoroacetyl)-2,3,4,5-tetrahydro-1,4-benzoxazepine

7-Nitro-2,3,4,5-tetrahydro-1,4-benzoxazepine (0.87 g, 4.5 mmol) and pyridine (1.4 ml, 18 mmol) were dissolved in dichloromethane (10 ml). Trifluoroacetic anhydride (0.90 ml, 6.75 mmol) was added dropwise at −10° C. under argon atmosphere. The mixture was stirred at 0° C. for 30 min and at ambient temperature for 1 h. Ice-water (2 ml) was added followed by dichloromethane (25 ml). The mixture was washed with water. The organic phase was dried (MgSO₄) and the solvent was evaporated to give the title compound that was used directly in the next step. MS EI m/z M⁺ 290.

### (vi) 4-(Trifluoroacetyl)-2,3,4,5-tetrahydro-1,4-benzoxazepin-7-amine

The crude 7-nitro-4-(trifluoroacetyl)-2,3,4,5-tetrahydro-1,4-benzoxazepine (from example 9v) was dissolved in EtOAc:methanol (1:1, 50 ml). 10% Palladium on charcoal (100 mg) was added and the mixture was hydrogenated at 50 psi pressure of hydrogen gas for 16 h. The mixture was filtered through Al₂O₃ (neutral) and celite. The solvent was removed to give the title compound (1.1 g, 94% over 2 steps). MS ESI m/z M+H⁺ 261.

### Example 10-14

Examples 10-14 were prepared according to the method in example 9(i) and the products were isolated as the acetate salt.

| Example | | | MS | |
|---|---|---|---|---|
| no | Name | 1H NMR (400 MHz) | ESI m/z | yield |
| 10 | 4-chloro-N-(2,3,4,5- | (CHLOROFORM-d) | M + H+ 389, | 88% |
| | tetrahydro-1,4- | δ ppm 8.67-8.73 (1H, | 391 | |
| | benzoxazepin-7- | m) 8.06 (1H, d) | | |
| | yl)naphthalene-1- | 7.68-7.75 (2H, m) | | |
| | sulfonamide | 7.57 (2H, d) | | |
| | | 6.69-6.82 (3H, m) | | |
| | | 3.97-4.04 (2H, m) 3.83 (2H, | | |
| | | s) 3.21-3.27 (2H, | | |
| | | m) | | |
| 11 | 4-fluoro-N-(2,3,4,5- | (CHLOROFORM-d) | M + H+ 373 | 69% |
| | tetrahydro-1,4- | δ ppm 8.67 (1H, d) | | |
| | benzoxazepin-7- | 8.21 (1H, d) 8.13 (1H, | | |
| | yl)naphthalene-1- | dd) 7.64-7.76 (2H, | | |
| | sulfonamide | m) 7.12 (1H, t) | | |
| | | 6.69-6.83 (3H, m) | | |
| | | 3.97-4.04 (2H, m) | | |
| | | 3.83 (2H, s) | | |
| | | 3.20-3.27 (2H, m) | | |
| 12 | 3-chloro-2-fluoro-N- | (CHLOROFORM-d) | M + H+ 357, | 80% |
| | (2,3,4,5-tetrahydro-1,4- | δ ppm 7.67-7.74 (1H, | 359 | |
| | benzoxazepin-7- | m) 7.54-7.61 (1H, | | |
| | yl)benzenesulfonamide | m) 7.16 (1H, t) | | |
| | | 7.01-7.09 (1H, m) | | |
| | | 6.97 (1H, br. s.) | | |
| | | 6.92 (1H, d) 4.10 (2H, | | |
| | | br. s.) 4.02 (2H, br. | | |
| | | s.) 3.35 (2H, br. s.) | | |
| 13 | 5-chloro-N-(2,3,4,5- | (CHLOROFORM-d) | M + H+ 389, | 54% |
| | tetrahydro-1,4- | δ ppm 8.29-8.36 (2H, | 391; M − H+ | |
| | benzoxazepin-7- | m) 7.79-7.85 (2H, | 387, 389 | |
| | yl)naphthalene-2- | m) 7.72 (1H, d) | | |
| | sulfonamide | 7.50 (1H, t) | | |
| | | 6.87-6.92 (3H, m) | | |
| | | 4.00-4.05 (2H, m) 3.89 (2H, | | |
| | | s) 3.21-3.27 (2H, | | |
| | | m) | | |
| 14 | 5-chloro-N-(2,3,4,5- | (CHLOROFORM-d) | M + H+ 389, | 34% |
| | tetrahydro-1,4- | δ ppm 8.62 (1H, d) | 391; M − H+ | |
| | benzoxazepin-7- | 8.57 (1H, d) 8.21 (1H, | 387, 389 | |
| | yl)naphthalene-1- | d) 7.72 (1H, d) | | |
| | sulfonamide | 7.54-7.62 (2H, m) | | |
| | | 6.79 (1H, d) 6.76 (1H, | | |
| | | d) 6.68 (1H, dd) | | |
| | | 3.95-4.01 (2H, m) | | |
| | | 3.81 (2H, s) | | |
| | | 3.17-3.24 (2H, m) | | |

### Example 15

### N-(2,3,4,5-Tetrahydro-1,4-benzoxazepin-7-yl)biphenyl-2-sulfonamide

4-(Trifluoroacetyl)-2,3,4,5-tetrahydro-1,4-benzoxazepin-7-amine (20 mg, 0.077 mmol), pyridine (12 μl, 0.15 mmol) and biphenyl-2-sulfonyl chloride (20 mg, 0.079 mmol) were dissolved in chloroform (1 ml). The reaction mixture was stirred at ambient temperature under nitrogen atmosphere for 4 h. Aqueous sodium hydroxide (2 M, 0.5 ml) was added and the stirring was continued for 1 h. The solvent was removed and the residue was purified by preparative HPLC to give a dry film (13 mg, 40%). ¹H NMR (400 MHz, METHANOL-d₄) δ ppm 8.10 (1H, d) 7.60 (1H, t) 7.51 (1H, t) 7.35-7.43 (3H, m) 7.21-7.30 (3H, m) 6.82 (1H, d) 6.67-6.76 (2H, m) 3.96-4.03 (2H, m) 3.86 (2H, s) 3.17-3.23 (2H, m); MS ESI m/z M+H⁺ 381, M−H⁺ 379.

### Example 16-21

Examples 16-21 were prepared according to the method in Example 15.

| | | | | state, |
|---|---|---|---|---|
| Example no | Name | 1H NMR (400 MHz) | MS m/z | yield |
| 16 | N-(2,3,4,5-tetrahydro-1,4- | (METHANOL- | AP | dry film, |
| | benzoxazepin-7- | d₄) δ ppm | M + H+ | 40% |
| | yl)biphenyl-3-sulfonamide | 7.88-7.92 (1H, m) | 381 | |
| | | 7.81-7.85 (1H, | | |
| | | m) 7.69-7.74 (1H, | | |
| | | m) | | |
| | | 7.52-7.60 (3H, m) | | |
| | | 7.43-7.50 (2H, m) | | |
| | | 7.37-7.43 (1H, | | |
| | | m) 7.04 (1H, d) | | |
| | | 6.96-7.00 (1H, | | |
| | | m) 6.92 (1H, d) | | |
| | | 3.99-4.04 (2H, | | |
| | | m) 3.95 (2H, s) | | |
| | | 3.20-3.26 (2H, | | |
| | | m) | | |
| 17 | N-(2,3,4,5-tetrahydro-1,4- | (METHANOL- | AP | dry film, |
| | benzoxazepin-7-yl)-2,3- | d₄) δ ppm | M + H+ | 77% |
| | dihydro-1-benzofuran-5- | 7.55-7.60 (1H, m) | 347 | |
| | sulfonamide | 7.50 (1H, dd) | | |
| | | 7.01 (1H, d) | | |
| | | 6.91-6.96 (1H, m) | | |
| | | 6.89 (1H, d) | | |
| | | 6.75 (1H, d) 4.62 (2H, | | |
| | | dd) | | |
| | | 4.00-4.06 (2H, m) 3.95 (2H, | | |
| | | s) | | |
| | | 3.17-3.27 (4H, m) | | |
| 18 | N-(2,3,4,5-tetrahydro-1,4- | (DMSO-d6) δ | AP | dry film, |
| | benzoxazepin-7- | ppm 9.14 (1H, | M + H+ | 67% |
| | yl)quinoline-8-sulfonamide | dd) 8.53 (1H, dd) | 356 | |
| | | 8.24-8.34 (2H, | | |
| | | m) 7.64-7.78 (2H, | | |
| | | m) 6.83 (1H, | | |
| | | d) 6.72 (1H, dd) | | |
| | | 6.67 (1H, d) | | |
| | | 3.74-3.79 (2H, m) | | |
| | | 3.53 (2H, s) | | |
| | | 2.85-2.92 (2H, m) | | |
| 19 | 1-(3-chlorophenyl)-N- | (DMSO-d6) δ | AP | solid, 21% |
| | (2,3,4,5-tetrahydro-1,4- | ppm | M + H+ | |
| | benzoxazepin-7- | 7.31-7.44 (3H, m) | 353, 355 | |
| | yl)methanesulfonamide | 7.20-7.26 (1H, m) | | |
| | | 6.86-6.98 (3H, | | |
| | | m) 4.45 (2H, s) | | |
| | | 3.87-3.94 (2H, | | |
| | | m) 3.72 (2H, s) | | |
| | | 2.97-3.04 (2H, | | |
| | | m) | | |
| 20 | N-(2,3,4,5-tetrahydro-1,4- | (METHANOL- | AP | dry film, |
| | benzoxazepin-7-yl)-5,6,7,8- | d₄) δ ppm | M + H+ | 11% |
| | tetrahydronaphthalene-2- | 7.38-7.45 (2H, m) | 359 | |
| | sulfonamide | 7.14 (1H, d) | | |
| | | 7.00 (1H, d) | | |
| | | 6.91-6.96 (1H, m) | | |
| | | 6.88 (1H, d) | | |
| | | 3.99-4.06 (2H, m) | | |
| | | 3.95 (2H, s) | | |
| | | 3.20-3.27 (2H, m) | | |
| | | 2.71-2.82 (4H, | | |
| | | m) 1.75-1.84 (4H, | | |
| | | m) | | |
| 21 | N-(2,3,4,5-tetrahydro-1,4- | (METHANOL- | AP | dry film, |
| | benzoxazepin-7-yl)-5,6,7,8- | d₄) δ ppm 7.74 (1H, | M + H+ | 11% |
| | tetrahydronaphthalene-1- | d) 7.28 (1H, | 359 | |
| | sulfonamide | d) 7.17 (1H, t) | | |
| | | 6.91-7.01 (2H, | | |
| | | m) 6.88 (1H, d) | | |
| | | 3.98-4.05 (2H, | | |
| | | m) 3.93-3.98 (2H, | | |
| | | m) | | |
| | | 3.21-3.28 (2H, m) | | |
| | | 3.11-3.19 (2H, m) | | |
| | | 2.78-2.86 (2H, | | |
| | | m) 1.73-1.88 (4H, | | |
| | | m) | | |

### Example 22

### (i): 2,3-Dichloro-N-[4-(2-fluoroethyl)-2,3,4,5-tetrahydro-1,4-benzoxazepin-7-yl]benzenesulfonamide

2,3-Dichlorobenzenesulfonyl chloride (15 mg, 0.060 mmol) was dissolved in chloroform (1 ml) and pyridine (10 μl, 0.12 mmol) and 4-(2-fluoroethyl)-2,3,4,5-tetrahydro-1,4-benzoxazepin-7-amine (13 mg, 0.060 mmol) were added. The mixture was stirred at ambient temperature for 1 h, methanol (1 ml) was added and the solvent was removed by evaporation. The residue was purified by preparative HPLC to give the title compound (13 mg, 50%).

¹H NMR (400 MHz, CHLOROFORM-d) δ ppm 7.91 (1H, d) 7.63 (1H, dd) 7.24-7.31 (1H, m) 6.90-6.97 (2H, m) 6.85 (1H, d) 4.59 (1H, t) 4.47 (1H, t) 3.95-4.00 (2H, m) 3.82 (2H, s) 3.10-3.15 (2H, m) 2.74 (1H, t) 2.67 (1H, t); MS ESI m/z M+H⁺ 419, 421.

### (ii) 4-(2-Fluoroethyl)-7-nitro-2,3,4,5-tetrahydro-1,4-benzoxazepine

2-Fluoroethanol (0.35 ml, 6.06 mmol) and triethylamine (1.0 ml, 7.28 mmol) were dissolved in dichloromethane (5 ml) and the mixture was cooled to −10° C. under nitrogen atmosphere. Mesylchloride (0.565 ml, 7.28 mmol) was added. The mixture was stirred at −10° C. for 30 min. Ice-water was added and the phases were separated. The organic phase was washed with 5% hydrochloric acid, saturated aqueous sodium hydrogen carbonate and water. The organic phase was dried (MgSO₄) and the solvent was evaporated. The crude was dissolved in DMF (6 ml) and 7-nitro-2,3,4,5-tetrahydro-1,4-benzoxazepine (0.98 g, 5.06 mmol) and DIPEA (0.87 ml, 5.06 mmol) was added. The mixture was heated at 90° C. for 18 h. The mixture was cooled to RT and ice-water (10 ml) was added. The mixture was is extracted with toluene (×1) and the organic phase was washed with water, dried (MgSO₄) and the solvent was evaporated. The residue was purified by preparative HPLC to give the title compound (0.265 g, 22%). MS ESI m/z M+H⁺ 241.

### (iii) 4-(2-Fluoroethyl)-2,3,4,5-tetrahydro-1,4-benzoxazepin-7-amine

4-(2-Fluoroethyl)-7-nitro-2,3,4,5-tetrahydro-1,4-benzoxazepine (0.265 g, 1.1 mmol) was dissolved in EtOAc (14 ml) and methanol (7 ml). 10% palladium on charcoal (50 mg) was added and the mixture was hydrogenated at 50 psi pressure of hydrogen gas for 16 h. The mixture was filtered through Al₂O₃ (neutral) and celite. The solvent was removed to give the title compound (0.169 g, 73%). MS ESI m/z [M+MeCN+H]⁺ 252.

### Example 23

### 4-Chloro-N-[4-(2-fluoroethyl)-2,3,4,5-tetrahydro-1,4-benzoxazepin-7-yl]naphthalene-1-sulfonamide

The method presented in example 22 (i) was used to prepare the title compound (23 mg, 69%). ¹NMR (400 MHz, CHLOROFORM-d) δ ppm 8.68-8.73 (1H, m) 8.39-8.44 (1H, m) 8.05 (1H, d) 7.69-7.76 (2H, m) 7.55 (1H, d) 6.71-6.80 (2H, m) 6.65 (1H, d) 4.53 (1H, t) 4.41 (1H, t) 3.93-3.98 (2H, m) 3.71 (2H, s) 3.08-3.14 (2H, m) 2.64 (1H, t) 2.57 (1H, t); MS ESI m/z M+H⁺ 435, 437.

### Example 24

### (i): 2,3-Dichloro-N-[(11aS)-2,3,11,11a-tetrahydro-1H,5H-pyrrolo[2,1-c][1,4]benzoxazepin-7-yl]benzenesulfonamide

(11aS)-2,3,11,11a-Tetrahydro-1H,5H-pyrrolo[2,1-c][1,4]benzoxazepin-7-amine (37 mg, 0.180 mmol) was dissolved in dichloromethane and PS-MEA (3.72 mmol/g, 150 mg) was added followed by 2,3-dichlorobenzensulfonyl chloride (55 mg, 0.225 mmol) in dichloromethane (0.5 ml). The mixture was shaken at ambient temperature for 20 h. PS-trisamine (2.35 mmol/g, 50 mg) was added as a slurry in dichloromethane and the mixture was shaken for 3 h. The resins were removed by filtration and washed with dichloromethane, methanol and THF. The solvents were removed by evaporation and the residue was purified by preparative HPLC to give the title compound (30 mg, 40%). ¹H NMR (400 MHz, CHLOROFORM-d) δ ppm 7.41 (dd, 1H) 7.65 (dd, 1H) 7.28 (t, 1H) 6.91-6.96 (m, 2H) 6.83-6.87 (m, 1H) 4.28 (dd, 1H) 3.68 (d, 1H) 3.56-3.63 (m, 1H) 3.43 (dd, 1H) 3.09-3.16 (m, 1H) 2.65-2.75 (m, 1H) 2.49 (q, 1H) 1.75-1.95 (m, 3H) 1.34-1.46 (m, 1H);

MS ESI m/z M+H⁺ 413, 415, 417; M−H⁺ 411, 413, 415.

### (ii) [(2S)-1-(2-Fluoro-5-nitrobenzyl)pyrrolidin-2-yl]methanol

To a solution of 2-fluoro-5-nitrobenzaldehyde (595 mg, 3.52 mmol) in THF (10 ml) was added a solution of (S)-(+)-2-(hydroxymethyl)pyrrolidine in THF (10 ml) followed by acetic acid (251 μl, 4.40 mmol). The mixture was cooled to 0° C. and sodium cyanoborohydride (276 mg, 4.40 mmol) was added. The cooling bath was removed and the mixture was stirred at RT for 16 h. The mixture was diluted with EtOAc and washed with saturated aqueous sodium hydrogen carbonate. The organic phase was dried (Na₂SO₄) and the solvent was evaporated. The residue was purified by column chromatography on silica eluting with a gradient of 0-7% methanol in dichloromethane to give an oil (629 mg, 70%). MS ESI m/z M+H⁺ 255.

### (iii)(11aS)-7-Nitro-2,3,11,11a-tetrahydro-1H,5H-pyrrolo[2,1-c][1,4]benzoxazepine

[(2S)-1-(2-Fluoro-5-nitrobenzyl)pyrrolidin-2-yl]methanol (620 mg, 2.44 mmol) was dissolved in anhydrous THF (15 ml) and sodium hydride (88 mg, 3.66 mmol) was added. The mixture was stirred at ambient temperature under argon atmosphere for 5 h. EtOAc was added and the mixture was washed with saturated aqueous sodium hydrogen carbonate. The organic phase was dried (Na₂SO₄) and the solvent was evaporated. The residue was purified by column chromatography on silica eluting with a gradient of 0-10% methanol in dichloromethane to give a solid (284 mg, 50%). MS ESI m/z M+H⁺ 235.

### (iv) (11aS)-2,3,11,11a-Tetrahydro-1H,5H-pyrrolo[2,1-c][1,4]benzoxazepin-7-amine

(11aS)-7-Nitro-2,3,11,11a-tetrahydro-1H,5H-pyrrolo[2,1-c][1,4]benzoxazepine (279 mg, 1.19 mmol) was dissolved in methanol (10 ml) and ammonia (7 M in methanol, 2 ml) was added followed by 10% palladium on charcoal (130 mg). The mixture was hydrogenated at atmospheric pressure of hydrogen gas for 2 h. The mixture was filtered through celite and the solvent was evaporated to give an oil (225 mg, 92%). MS ESI m/z M+H⁺ H 205.

### Examples 25-27

Examples 25-27 were prepared according to the method in example 24(i)

| Example | | | MS ESI | |
|---|---|---|---|---|
| no | Name | 1H NMR (400 MHz) | m/z | state, yield |
| 25 | N-[(11aS)-2,3,11,11a- | CHLOROFORM-d) | M + H+ | 16 mg, 22% |
| | tetrahydro-1H,5H-pyrrolo[2,1- | δ ppm 8.66 (1H, d) | 395; M − H+ | |
| | c][1,4]benzoxazepin-7- | 8.13 (1H, d) 8.03 (1H, | 393 | |
| | yl]naphthalene-1-sulfonamide | d) 7.95 (1H, d) | | |
| | | 7.58-7.71 (2H, m) | | |
| | | 7.45 (1H, t) 6.76 (1H, | | |
| | | d) 6.72 (1H, d) | | |
| | | 6.64 (1H, dd) | | |
| | | 4.24 (1H, dd) 3.55 (2H, | | |
| | | s) 3.37-3.45 (1H, | | |
| | | m) 3.02-3.10 (1H, | | |
| | | m) 2.66-2.75 (1H, | | |
| | | m) 2.45 (1H, q) | | |
| | | 1.74-1.93 (3H, m) | | |
| | | 1.32-1.45 (1H, m) | | |
| 26 | N-[(11aS)-2,3,11,11a- | δ ppm 7.78 (2H, d) | M + H+ | 24 mg, 32% |
| | tetrahydro-1H,5H-pyrrolo[2,1- | 7.64 (2H, d) | 421; M − H+ | |
| | c][1,4]benzoxazepin-7- | 7.56-7.60 (2H, m) | 419 | |
| | yl]biphenyl-4-sulfonamide | 7.39-7.50 (3H, m) 6.93 (1H, | | |
| | | br. s.) 6.85 (2H, | | |
| | | br. s.) 4.29 (1H, dd) | | |
| | | 3.65 (2H, q) 3.47 (1H, | | |
| | | dd) 3.10-3.18 (1H, | | |
| | | m) 2.73 (1H, q) | | |
| | | 2.48 (1H, q) | | |
| | | 1.74-1.95 (3H, m) | | |
| | | 1.35-1.47 (1H, m) | | |
| 27 | N-[(11aS)-2,3,11,11a- | δ ppm 7.99 (1H, s) | M + H+ | 10 mg, 14% |
| | tetrahydro-1H,5H-pyrrolo[2,1- | 7.87 (1H, d) 7.79 (1H, | 413; M − H+ | |
| | c][1,4]benzoxazepin-7-yl]-3- | d) 7.58 (1H, t) | 411 | |
| | (trifluoromethyl)benzenesulfonamide | 6.77-6.91 (3H, m) | | |
| | | 4.30 (1H, dd) | | |
| | | 3.60-3.71 (2H, m) 3.49 (1H, | | |
| | | dd) 3.10-3.18 (1H, | | |
| | | m) 2.74-2.83 (1H, | | |
| | | m) 2.51 (1H, q) | | |
| | | 1.77-1.97 (3H, m) | | |
| | | 1.38-1.49 (1H, m) | | |

### Example 28

### (i): 3-Bromo-N-[9-(trifluoromethyl)-2,3,4,5-tetrahydro-1,4-benzoxazepin-7-yl]benzenesulfonamide

9-(Trifluoromethyl)-2,3,4,5-tetrahydro-1,4-benzoxazepin-7-amine (23 mg, 0.10 mmol) was dissolved in dioxane (1 ml). Hydrogen chloride (2M in ether, 75 μl, 0.15 mmol) was added and a precipitate was formed. 3-Bromobenzenesulfonyl chloride (14.4 μl, 0.10 mmol) was added and after 10 minutes DIPEA (9 μl. 0.05 mmol) was added. Acetonitrile (0.25 ml) was added and the mixture was heated until the precipitate had dissolved. The mixture was stirred at ambient temperature and after 20 min DIPEA (9 μl, 0.05 mmol) was added. The mixture was stirred for 15 min and DIPEA (9 μl, 0.05 mmol) was added. The mixture was stirred for 10 min and DIPEA (9 μl, 0.05 mmol) was added. The mixture was stirred for additional 10 min and DIPEA (9 μl, 0.05 mmol) was added. Water (200 μl) was added and the mixture was purified by preparative HPLC to give the title compound (10.5 mg, 23%). ¹H NMR (400 MHz, CHLOROFORM-d) δ ppm 7.86-7.89 (1H, m) 7.64-7.69 (2H, m) 7.43 (1H, br. s.) 7.33 (1H, t) 7.22 (1H, d) 7.18 (1H, d) 4.68 (2H, s) 3.77-3.82 (2H, m) 3.14-3.19 (2H, m); MS ESI m/z M+H⁺ 451, 453.

### (ii) 2-Fluoro-5-nitro-3-(trifluoromethyl)benzaldehyde

2-Fluoro-3-(trifluoromethyl)benzaldehyde (2.76 ml, 20 mmol) was dissolved in concentrated sulfuric acid (5 ml) and concentrated nitric acid was added dropwise. The mixture was stirred at ambient temperature for 30 min. The mixture was poured onto water and was extracted with dichlormethane (×3). The combined organic layers were washed with aqueous sodium hydroxide (1M) and the aqueous layer was extracted with dichloromethane (×2). All organic layers were combined, dried (Na₂SO₄) and the solvents were evaporated to give the title compound (2.73 g, 58%). ¹H NMR (400 MHz, CHLOROFORM-d) δ ppm 10.43 (1H, s) 8.93-8.99 (1H, m) 8.74-8.81 (1H, m); MS EI m/z M⁺ 237.

### (iii) 9-(Trifluoromethyl)-2,3,4,5-tetrahydro-1,4-benzoxazepin-7-amine

2-Fluoro-5-nitro-3-(trifluoromethyl)benzaldehyde (474 mg, 2.0 mmol) was dissolved in anhydrous dioxane (5 ml) and 2-aminoethanol (133 μl, 2.2 mmol) and molecular sieves 3 Å were added. The mixture was stirred at ambient temperature for 20 h. Sodium cyanoborohydride (113 mg, 3.0 mmol) was added and the mixture was stirred for 24 h. Palladium black (50 mg), ammonium formate (1 g) and methanol (5 ml) was added. The mixture was stirred for 1 h and additional ammonium formate (1 g) was added. The mixture was stirred for 5 h at ambient temperature and the mixture was filtered through celite and the celite was washed with methanol. The filtrate was concentrated and the residue was dissolved in EtOAc and washed with water. The aquoeous layer was made basic with 1M aqueous sodium hydroxide (pH 10) and was extracted with EtOAc. The combined organic layers were dried (Na₂SO₄) and the solvent was evaporated. The residue was purified by column chromatography on silica eluting with EtOAc:methanol 9:1 to give the title compound (220 mg, 47%). ¹H NMR (400 MHz, CHLOROFORM-d) δ ppm 6.82 (1H, d) 6.78 (1H, d) 4.70 (2H, s) 3.78-3.83 (2 II, m) 3.05-3.10 (2H, m); MS ESI m/z M+H 233.

### Example 29

### 5-Chloro-3-methyl-N-[9-(trifluoromethyl)-2,3,4,5-tetrahydro-1,4-benzoxazepin-7-yl]-1-benzothiophene-2-sulfonamide

9-(Trifluoromethyl)-2,3,4,5-tetrahydro-1,4-benzoxazepin-7-amine (23 mg, 0.10 mmol) was dissolved in dioxane (0.75 ml). Hydrogen chloride (2M in ether, 75 μl, 0.15 mmol) was added followed by acetonitrile (0.25 ml) and a precipitated was formed. 5-Chloro-3-methylbenzo[B]thiophene-2-sulfonyl chloride (28 mg, 0.10 mmol) was added followed by DIPEA (25 μl). The mixture was heated until the precipitate had dissolved. DIPEA (10 μl) was added and after 10 min additional DIPEA (10 μl) was added. The mixture was stirred at ambient temperature for 20 h and the reaction mixture was filtered through a plug of silica. The silica was washed with methanol (0.5 ml). The filtrate was purified by column chromatography on silica eluting with heptane:EtOAc (1:2) to give the title compound (10 mg, 20%). ¹H NMR (400 MHz, CHLOROFORM-d) δ ppm 7.48 (1H, d) 7.44 (1H, d) 7.11-7.17 (2H, m) 7.06 (1H, d) 4.34 (2H, d)) 3.41-3.48 (2H, m) 2.80-2.87 (2H, m) 2.16 (3H, s); MS ESI m/z M+H⁺ 477, 479.

### Example 30-33

Examples 30-33 was prepared according to the method in example 29.

| Example | | | | |
|---|---|---|---|---|
| no | Name | 1H NMR (400 MHz) | MS m/z | state, yield |
| 30 | N-[4-methyl-5-({[9- | (ACETONE-d6) δ | M + H+ 451 | dry film, 12 mg, |
| | (trifluoromethyl)-2,3,4,5- | ppm 7.58 (1H, d) | | 27% |
| | tetrahydro-1,4- | 7.38 (1H, d) | | |
| | benzoxazepin-7- | 4.67 (2H, s) | | |
| | yl]amino}sulfonyl)-1,3- | 3.69-3.74 (2H, m) | | |
| | thiazol-2-yl]acetamide | 3.10-3.16 (2H, m) | | |
| | | 2.24 (6H, s) | | |
| 31 | 2,3-dichloro-N-[9- | (CHLOROFORM- | M + H+ 441, | dry film, 14 mg, |
| | (trifluoromethyl)-2,3,4,5- | d) δ ppm 7.91 (1H, | 443 | 33% |
| | tetrahydro-1,4- | dd) 7.61 (1H, | | |
| | benzoxazepin-7- | dd) 7.34 (1H, d) | | |
| | yl]benzenesulfonamide | 7.29 (1H, d) | | |
| | | 7.24-7.26 (1H, m) | | |
| | | 4.58 (2H, s) | | |
| | | 3.66-3.71 (2H, m) | | |
| | | 3.03-3.08 (2H, m) | | |
| 32 | 3-(trifluoromethyl)-N-[9- | (CHLOROFORM- | M + H+ 441 | dry film 14 mg, |
| | (trifluoromethyl)-2,3,4,5- | d) δ ppm | | 31% |
| | tetrahydro-1,4- | 7.92-7.97 (2H, m) | | |
| | benzoxazepin-7- | 7.82 (1H, d) 7.62 (1H, | | |
| | yl]benzenesulfonamide | t) 7.30 (1H, br. s.) | | |
| | | 7.22 (1H, d) | | |
| | | 7.14 (1H, d) 4.68 (2H, | | |
| | | s) 3.78-3.83 (2H, | | |
| | | m) 3.13-3.19 (2H, | | |
| | | m) | | |
| 33 | N-[9-(trifluoromethyl)- | (CHLOROFORM- | M + H+ 423 | dry film, 14 mg, |
| | 2,3,4,5-tetrahydro-1,4- | d) δ ppm 8.60 (1H, | | 33% |
| | benzoxazepin-7- | d) | | |
| | yl]naphthalene-1- | 8.09-8.15 (1H, m) 7.96 (1H, | | |
| | sulfonamide | d) | | |
| | | 7.81-7.88 (1H, m) | | |
| | | 7.46-7.56 (2H, m) | | |
| | | 7.34-7.44 (1H, m) | | |
| | | 7.16 (1H, d) | | |
| | | 7.05 (1H, d) 4.49 (2H, | | |
| | | s) 3.62-3.68 (2H, | | |
| | | m) 2.96-3.03 (2H, | | |
| | | m) | | |

### Example 34

### (i): 2,3-Dichloro-N-(2,3,4,5-tetrahydro-1H-2-benzazepin-8-yl)benzenesulfonamide

2-(Trifluoroacetyl)-2,3,4,5-tetrahydro-1H-2-benzazepin-8-amine (69 mg, 0.27 mmol) and pyridine (43 μl, 0.54 mmol) were dissolved in chloroform (1 ml) and acetonitrile (1 ml). 2,3-Dichlorobenzenesulfonyl chloride (79 mg, 0.32 mmol) was added and the mixture was stirred at ambient temperature for 1 h. Water (1 ml) was added and the solvent was evaporated. The residue was dissolved in chloroform (2.5 ml) and aqueous sodium hydroxide (2M, 2.5 ml) was added. The mixture was stirred at ambient temperature for 40 min. The mixture was diluted with water (8 ml) and hydrochloric acid was added until acidic pH. Sodium hydrogen carbonate was added until basic pH and the mixture was extracted with chloroform. The organic phase was dried (MgSO₄) and the solvent was evaporated. The residue was purified by preparative HPLC to give the acetate of the title compound as a solid (32 mg, 32%). ¹H NMR (400 MHz, METHANOL-d₄) δ ppm 8.04 (1H, dd) 7.76 (1H, dd) 7.41 (1H, t) 7.04-7.15 (3H, m) 4.16-4.20(2H, m) 3.33-3.38 (2H, m) 2.90-2.96 (2H, m) 1.86 (2H, br. s.); MS ESI m/z M+H⁺ 371.

### (ii) 8-Nitro-2,3,4,5-tetrahydro-1H-2-benzazepin-1-one

7-Nitro-1-tetralone (3.0 g, 15.7 mmol) and sodium azide (3.0 g, 47.0 mmol) were dissolved to in acetic acid (31 ml) and the mixture was cooled to 0° C. Concentrated sulfuric acid (5 ml) was added dropwise. The mixture was heated at 50° C. for 4 h and cooled to RT. The mixture was poured onto ice (50 ml) and the mixture was basified with concentrated ammonium hydroxide. The mixture was stirred at ambient temperature for 20 h and the solid formed was collected by filtration. The product was isolated by preparative HPLC to give the title compound (0.51 g, 16%). 1H NMR (400 MHz, DMSO-d6) δ ppm 8.36 (1H, br. s.) 8.21-8.29 (2H, m) 7.57 (1H, d) 2.94 (2H, q) 2.87 (2H, t) 1.89-1.99 (2H, m); MS PSI m/z M+H+ 207.

### (iii) 8-Nitro-2,3,4,5-tetrahydro-1H-2-benzazepine

8-Nitro-2,3,4,5-tetrahydro-1H-2-benzazepin-1-one (330 mg, 1.60 mmol) was suspended in THF (2 ml) and borane (1M in THF, 6.5 ml, 6.5 mmol) was added. The mixture was refluxed for 4 h, cooled to 0° C. and hydrochloric acid (4 M, 3 ml) was added carefully. The mixture was refluxed for 1 h, the solvents were evaporated and water (11 ml) was added. The mixture was neutralized by addition of solid sodium hydrogen carbonate. The mixture was extracted with EtOAc (×3). The organic phase was dried (MgSO₄) and the solvent was evaporated. The residue was purified by column chromatography on silica eluting with 10% methanol in chloroform containg 0.1% triethylamine to give the title compound (258 mg, 84%). MS ESI m/z M+H⁺ 193.

### (iv) 8-Nitro-2-(trifluoroacetyl)-2,3,4,5-tetrahydro-1H-2-benzazepine

8-Nitro-2,3,4,5-tetrahydro-1H-2-benzazepine (258 mg, 1.34 mmol) was dissolved in dichloromethane (5 ml) and pyridine (0.43 ml, 5.2 mmol) was added. The mixture was cooled to 0° C. and trifluoroacetic anhydride (0.28 ml, 2.01 mmol) was added dropwise. The mixture was stirred at 0° C. for 30 min and at RT for 1 h. Water (1 ml) was added followed by dichloromethane (25 ml). The mixture was washed with water, the organic phase was dried (MgSO₄) and the solvent was evaporated to give the title compound (346 mg, 89%). MS ESI m/z M−H⁺ 287.

### (v) 2-(Trifluoroacetyl)-2,3,4,5-tetrahydro-1H-2-benzazepin-8-amine

8-Nitro-2-(trifluoroacetyl)-2,3,4,5-tetrahydro-1H-2-benzazepine (346 mg, 1.20 mmol) was dissolved in EtOAc (3 ml) and methanol (3 ml). Ammonia (7 M in methanol, 2 drops) was added followed by 10% palladium on charcoal (50 mg). The mixture was hydrogenated at atmospheric pressure of hydrogen gas for 8 h. The mixture was filtered through Al₂O₃/celite and the solvent was evaporated to give the title compound (346 mg, quantitative yield). MS ESI m/z M+H⁺ 259.

### Example 35

### 4-Chloro-N-(2,3,4,5-tetrahydro-1H-2-benzazepin-8-yl)naphthalene-1-sulfonamide

The method presented in example 34 was used to give the acetate of the title compound as a dry film (25 mg, 21%). ¹H NMR (400 MHz, METHANOL-d₄) δ ppm 8.83 (1H, d) 8.36 (1H, d) 8.18 (1H, d) 7.71-7.83 (2H, m) 7.66 (1H, d) 6.93-7.06 (3H, m) 4.13 (2H, s) 3.31-3.32 (2H, m) 2.83-2.91 (2H, m) 1.83 (2H, br. s.); MS ESI m/z M+H⁺ 387, 389.

### Example 36

### N-(2,3,4,5-Tetrahydro-1H-2-benzazepin-8-yl)biphenyl-2-sulfonamide

The method presented in example 34 was used to give the acetate of the title compound as a dry film (32 mg, 24%). ¹H NMR (400 MHz, CHLOROFORM-d) δ ppm 8.17 (1H, d) 7.55 (1H, t) 7.36-7.51 (6H, m) 7.24-7.30 (1H, m) 6.95 (1H, d) 6.67-6.74 (1H, m) 6.56 (1H, s) 3.93 (2H, s) 3.20-3.28 (2H, m) 2.80-2.86 (2H, m) 1.80 (2H, br. s.); MS ESI m/z M+H⁺ 379.

### Example 37

### N-(2,3,4,5-Tetrahydro-1H-2-benzazepin-8-yl)quinoline-8-sulfonamide

The method presented in example 34 was used to give the acetate of the title compound as a solid (87 mg, 67%). ¹H NMR (400 MHz, CHLOROFORM-d) δ ppm 9.16 (1H, dd) 8.27-8.35 (2H, m) 8.03 (1H, dd) 7.55-7.64 (2 m) 6.88-6.93 (3H, m) 3.94 (2H, s) 3.21-3.28 (2H, m) 2.76-2.82 (2H, m) 1.80 (2H, br. s.); MS ESI m/z M+H⁺ 355.

### Example 38

### 1-[3-Chloro-5-(trifluoromethyl)pyridin-2-yl]-N-(2,3,4,5-tetrahydro-1H-2-benzazepin-8-yl)-1H-pyrrole-2-sulfonamide

2-(Trifluoroacetyl)-2,3,4,5-tetrahydro-1H-2-benzazepin-8-amine (34 mg, 0.13 mmol) and pyridine (22 μl, 0.26 mmol) were dissolved in chloroform (1 ml). 1-[3-Chloro-5- (trifluoromethyl)-2-pyridinyl]-1H-pyrrole-2-sulfonyl chloride (59 mg, 0.17 mmol) was added and the mixture was stirred at ambient temperature for 1 h. The solvent was evaporated and the residue was dissolved in methanol (0.5 ml) and ammonia (7 M in methanol, 3 ml) was added. The mixture was stirred at ambient temperature for 20 h. The solvents were evaporated and the residue was purified by preparative HPLC to give a dry film (25 mg, 40%).

¹H NMR (400 MHz, CHLOROFORM-d) δ ppm 8.62-8.65 (1H, m) 8.09 (1H, d) 7.90-7.92 (1H, m) 7.41 (1H, dd) 7.14-7.20 (2H, m) 7.05 (1H, d) 7.01 (1H, d) 6.55 (1H, dd) 4.04 (2H, s) 3.26-3.32 (2H, m) 2.84-2.91 (2H, m) 1.81-1.89 (2H, m); MS ESI m/z M+H⁺ 472.

### Example 39-49

Examples 39-49 were prepared according to the method in example 38.

| Example | | | MS ESI | |
|---|---|---|---|---|
| no | Name | 1HNMR (400 MHz) | m/z | state, yield |
| 39 | 4-phenyl-N-(2,3,4,5- | (CHLOROFORM-d) | M + H+ | dry film, 50% |
| | tetrahydro-1H-2-benzazepin- | δ ppm 8.25 (1H, s) | 453 | |
| | 8-yl)-5- | 7.38-7.46 (3H, m) | | |
| | (trifluoromethyl)thiophene-3- | 7.25-7.29 (2H, m) | | |
| | sulfonamide | 6.99 (1H, d) 6.80 (1H, | | |
| | | dd) 6.76 (1H, d) | | |
| | | 3.91 (2H, s) | | |
| | | 3.13-3.19 (2H, m) | | |
| | | 2.81-2.87 (2H, m) | | |
| | | 1.76-1.82 (2H, m) | | |
| 40 | 4′-chloro-N-(2,3,4,5- | (CHLOROFORM-d) | M + H+ | dry film, 87% |
| | tetrahydro-1H-2-benzazepin- | δ ppm 8.04-8.09 (2H, | 414 | |
| | 8-yl)biphenyl-2-sulfonamide | m) 7.45-7.51 (1H, | | |
| | | m) 7.28-7.33 (2H, | | |
| | | m) 7.13-7.21 (3H, | | |
| | | m) 6.86 (1H, d) | | |
| | | 6.49-6.56 (2H, m) | | |
| | | 3.67 (2H, s) | | |
| | | 3.01-3.08 (2H, m) | | |
| | | 2.71-2.77 (2H, m) | | |
| | | 1.55-1.63 (2H, m) | | |
| 41 | 4-(phenylsulfonyl)-N-(2,3,4,5- | (CHLOROFORM-d) | M + H+ | dry film, 56% |
| | tetrahydro-1H-2-benzazepin- | δ ppm 8.13 (1H, d) | 450 | |
| | 8-yl)thiophene-2-sulfonamide | 7.86-7.90 (2H, m) | | |
| | | 7.60-7.63 (2H, m) | | |
| | | 7.50-7.54 (2H, m) | | |
| | | 7.11 (1H, dd) 7.04 (1H, | | |
| | | d) 6.88-6.91 (1H, | | |
| | | m) 4.03-4.07 (2H, | | |
| | | m) 3.32 (2H, br. | | |
| | | s.) 2.85-2.94 (2H, | | |
| | | m) 1.91 (2H, br. s.) | | |
| 42 | 5-chloro-3-methyl-N-(2,3,4,5- | (CHLOROFORM-d) | M + H+ | 72% |
| | tetrahydro-1H-2-benzazepin- | δ ppm 7.63 (1H, s) | 408 | |
| | 8-yl)-1-benzothiophene-2- | 7.59 (1H, d) 7.32 (1H, | | |
| | sulfonamide | d) 7.21 (1H, dd) | | |
| | | 7.05 (1H, dd) 7.01 (1H, | | |
| | | d) 4.08 (2H, s) | | |
| | | 3.27-3.33 (2H, m) | | |
| | | 2.81-2.87 (2H, m) | | |
| | | 2.47 (3H, s) 1.88 (2H, | | |
| | | br. s.) | | |
| 43 | 2-bromo-N-(2,3,4,5- | (CHLOROFORM-d) | M + H+ | 75% |
| | tetrahydro-1H-2-benzazepin- | δ ppm 7.97 (2H, dd) | 382 | |
| | 8-yl)benzenesulfonamide | 7.58 (1H, dd) | | |
| | | 7.22-7.31 (1H, m) | | |
| | | 6.97-7.01 (2H, m) | | |
| | | 6.89-6.93 (1H, m) 4.01 (2H, | | |
| | | s) 3.20-3.26 (2H, | | |
| | | m) 2.73-2.78 (2H, | | |
| | | m) 1.75-1.82 (2H, | | |
| | | m) | | |
| 44 | N-(2,3,4,5-tetrahydro-1H-2- | (CHLOROFORM-d) | M + H+ | 60% |
| | benzazepin-8-yl)-2- | δ ppm 8.08 (1H, d) | 371 | |
| | (trifluoromethyl)benzenesulfonamide | 7.85 (1H, d) | | |
| | | 7.59-7.67 (2H, m) 7.09 (1H, | | |
| | | dd) 7.04 (1H, d) | | |
| | | 7.00 (1H, d) 4.09 (2H, | | |
| | | s) 3.30-3.36 (2H, | | |
| | | m) 2.85-2.91 (2H, | | |
| | | m) 1.80 (2H, br. | | |
| | | s.) | | |
| 45 | 2-iodo-N-(2,3,4,5-tetrahydro- | (CHLOROFORM-d) | M + H+ | dry film, 11% |
| | 1H-2-benzazepin-8- | δ ppm 8.09 (1H, dd) | 429 | |
| | yl)benzenesulfonamide | 7.99-8.05 (1H, m) | | |
| | | 7.42 (1H, t) | | |
| | | 7.12-7.18 (1H, m) | | |
| | | 6.99-7.09 (3H, m) 4.08 (2H, | | |
| | | br. s.) 3.32 (2H, | | |
| | | br. s.) 2.82-2.88 (2H, | | |
| | | m) 1.80 (2H, br. | | |
| | | s.) | | |
| 46 | 2,6-dichloro-N-(2,3,4,5- | (CHLOROFORM-d) | M + H+ | dry film, 20% |
| | tetrahydro-1H-2-benzazepin- | δ ppm 7.42 (2H, d) | 372 | |
| | 8-yl)benzenesulfonamide | 7.30 (1H, dd) 7.11 (1H, | | |
| | | dd) 7.05 (1H, d) | | |
| | | 7.00 (1H, d) 4.01 (2H, | | |
| | | s) 3.24-3.30 (2H, | | |
| | | m) 2.84-2.89 (2H, | | |
| | | m) 1.83 (2H, br. | | |
| | | s.) | | |
| 47 | N-(2,3,4,5-tetrahydro-1H-2- | (CHLOROFORM-d) | 387 | dry film, 46% |
| | benzazepin-8-yl)-2- | δ ppm 7.96 (1H, dd) | | |
| | (trifluoromethoxy)benzenesulfonamide | 7.54-7.60 (1H, m) | | |
| | | 7.30-7.39 (2H, m) | | |
| | | 6.99-7.06 (2H, m) | | |
| | | 6.93-6.97 (1H, m) | | |
| | | 3.99-4.02 (2H, m) | | |
| | | 3.23-3.29 (2H, m) | | |
| | | 2.82-2.88 (2H, m) | | |
| | | 1.80 (2H, br. s.) | | |
| 48 | 3,4-dichloro-N-(2,3,4,5- | (DMSO-d6) δ ppm | 372 | solid, 20% |
| | tetrahydro-1H-2-benzazepin- | 7.85 (1H, d) 7.73 (1H, | | |
| | 8-yl)benzenesulfonamide | d) 7.63 (1H, dd) | | |
| | | 6.90 (1H, d) 6.76 (1H, | | |
| | | d) 6.72 (1H, dd) | | |
| | | 3.76 (2H, s) | | |
| | | 3.02-3.07 (2H, m) 2.74 (2H, | | |
| | | br. s.) 1.58 (2H, | | |
| | | br. s.) | | |
| 49 | N-(2,3,4,5-tetrahydro-1H-2- | (CHLOROFORM-d) | 379 | dry film, 20% |
| | benzazepin-8-yl)biphenyl-4- | δ ppm 7.83 (2H, d) | | |
| | sulfonamide | 7.61 (2H, d) | | |
| | | 7.52-7.56 (2H, m) | | |
| | | 7.37-7.47 (3H, m) 7.12 (1H, | | |
| | | dd) 7.05 (1H, d) | | |
| | | 7.00 (1H, d) 4.05 (2H, | | |
| | | s) 3.27-3.33 (2H, | | |
| | | m) 2.85-2.90 (2H, | | |
| | | m) 1.80 (2H, br. | | |
| | | s.) | | |

### Example 50

### (i): 2,3-Dichloro-N-(2-methyl-2,3,4,5-tetrahydro-1H-2-benzazepin-8-yl)benzenesulfonamide

2-Methyl-2,3,4,5-tetrahydro-1H-2-benzazepin-8-amine (13 mg, 0.070 mmol) was dissolved in dichloromethane (1 ml) and pyridine (12 μl, 0.14 mmol) was added followed by 2,3-dichlorobenzenesulfonyl chloride (19 mg, 0.077 mmol) in dichloromethane (0.5 ml). The mixture was stirred at ambient temperature for 16 h and then diluted with dichloromethane (30 ml). The mixture was washed with aqueous sodium hydroxide and water. The organic layer was dried (MgSO₄) and the solvent was evaporated. The residue was purified by preparative HPLC to give the title compound (3 mg, 11%). ¹H NMR (400 MHz, METHANOL-d₄) δ ppm 7.99 (1H, dd) 7.72 (1H, dd) 7.37 (1H, t) 6.94-6.98 (3H, m) 3.69 (2H, s) 2.91-2.95 (2H, m) 2.76-2.81 (2H, m) 2.19 (3H, s) 1.64-1.71 (2H, m); MS ESI m/z M+H⁺ 385, 387, 389.

### (ii) 2-Methyl-8-nitro-2,3,4,5-tetrahydro-1H-2-benzazepine

8-Nitro-2,3,4,5-tetrahydro-1H-2-benzazepine (50 mg, 0.26 mmol) was dissolved in methanol (2 ml) and formaldehyde (37% aqueous solution, 97 μl, 1.3 mmol) and acetic acid (30 μl) were added. The mixture was stirred at ambient temperature for 1 h and sodium cyanoborohydride (49 mg, 0.78 mmol) was added. The mixture was stirred at ambient temperature for 16 h and the solvent was evaporated. The residue was dissolved in dichloromethane and was washed with aqueous sodium hydroxide and water. The organic phase was dried (MgSO₄) and the solvent was evaporated to give the title compound (47 mg, 88%). MS ESI m/z M+H⁺ 207.

### (iii) 2-Methyl-2,3,4,5-tetrahydro-1H-2-benzazepin-8-amine

Palladium on charcoal (10%, 6 mg) and ammonium formate (25 mg, 0.40 mmol) were mixed in ethanol (1 ml) under argon atmosphere. The mixture was stirred for 2 min and 2-methyl-8-nitro-2,3,4,5-tetrahydro-1H-2-benzazepine (20 mg, 0.10 mmol) was added and the vial was sealed. The mixture was heated at 140° C. for 15 min by microwave irradiation. The mixture was filtered through celite and the celite was washed with methanol. The solvent was evaporated to give the title compound (26 mg, quantitative yield). MS ESI m/z M+H⁺ 177.

### Example 51

### 4-Chloro-N-(2-methyl-2,3,4,5-tetrahydro-1H-2-benzazepin-8-yl)naphthalene-1-sulfonamide

The title compound was prepared according to the method presented in example 50(i) to give a solid (5 mg, 18%). ¹H NMR (400 MHz, METHANOL-d₄) δ ppm 8.83 (1H, dd) 8.35-8.39 (1H, m) 8.11 (1H, d) 7.72-7.81 (2H, m) 7.63 (1H, d) 6.90 (1H, d) 6.84 (1H, dd) 6.74 (1H, d) 3.60 (2H, s) 2.87-2.93 (2H, m) 2.70-2.76 (2H, m) 2.08 (3H, s) 1.59-1.66 (2H, m); MS ESI m/z M+H⁺ 401, 403, 404.

### Example 52

### 6-chloro-N-(2,3,4,5-tetrahydro-1H-2-benzazepin-8-yl)imidazo[2,1-b][1,3]thiazole-5-sulfonamide

6-chloroimidazo[2,1-b][1,3]thiazole-5-sulfonyl chloride (32 mg, 0.122 mmol) was added to a solution of 2-(trifluoroacetyl)-2,3,4,5-tetrahydro-1H-2-benzazepin-8-amine (21 mg, 0.081 mmol) and pyridine (13 μl, 0.16 mmol) in chloroform (2 ml). The reaction mixture was stirred for 1 h. Water (0.3 ml) was added and the solvent was removed in vacuum. The residue was redissolved in chloroform (2.5 ml) and sodium hydroxide solution (2 N, 2.5 ml). The mixture was stirred vigorously for 40 min. Water (10 ml) was added and the mixture was made acidic with conc hydrochloric acid and then made basic using solid NaHCO₃. The mixture was extracted three times with chloroform and the combined organic layers were dried over magnesium sulfate and concentrated. The residue was purified by preparative HPLC to give the title compound (1.28 mg 3.30%). ¹H NMR (400 MHz, CHLOROFORM-d) δ ppm 7.83 (1H, d) 6.97-7.06 (3H, m) 6.82 (1H, d) 3.88 (2H, s) 3.17 (2H, s) 2.81 (2H, d) 1.74 (2H, s); MS (ESI) m/z M+H⁺ 383 and 385.

### Example 53

### 2-Benzoyl-4-chloro-N-(2,3,4,5-tetrahydro-1H-2-benzazepin-8-yl)benzenesulfonamide

The compound was prepared according to the method presented in example 52 to give a solid (25%)

¹H NMR (400 MHz, CHLOROFORM-d) δ ppm 7.85 (2H, d) 7.65-7.76 (2H, m) 7.52 (2H, t) 7.41-7.47 (1H, m) 7.36 (1H, d) 7.05-7.16 (3H, m) 4.08 (2H, s) 3.30-3.37 (2H, m) 2.88-2.94 (2H, m) 1.90 (2H, m); MS (ESI) m/z M+H⁺ 440 and 442.

Pharmacology

Method for [125I]SB258585 Binding to Rat Striatal 5-HT6 Receptors

Materials

[¹²⁵I]SB258585 (1) with specific radioactivity 2000 Ci/mmol was purchased from Amersham Biosciences Europe GmbH, Freiburg, Germany. Other chemicals were purchased from commercial sources and were of analytical grade.

Preparation of Membranes

Striatal tissue from adult rats (Sprague-Dawley, 320-370 g, B & K Sweden) were dissected out, weighed and homogenized in buffer containing 50 mM Tris-HCl, 4 mM MgCl2, 1 mM EDTA, 10 μM pargyline and protease inhibitor (Complete, Roche Diagnostics) pH 7.4 using an Ultra-Turrax T8 (IKA Labortechnik, Germany). The tissue homogenate was centrifuged at 48 000×g for 10 min and the pellet was resuspended and recentrifuged as above. The final membranes were diluted in buffer to a concentration of 60 mg original wet weight (w.w.) per ml and stored in aliquots at −70° C.

Radioligand Binding Assays

Saturation binding studies were carried out in duplicate with 1-3 mg w.w. per tube in 0.5 ml buffer (50 mM Tris, 4 mM MgCl2, 100 mM NaCl, 1 mM EDTA, 5 mM ascorbate and 10 μM pargyline at pH 7.4), 0.2 nM [¹²⁵I]SB258585 and unlabelled SB258585 to give a final concentration range of 0.23-20 nM (12 conc.). Non-specific binding was determined in the presence of 10 μM methiothepin. In the competition experiments 0.8-2 mg w.w. per tube and a radioligand concentration of 0.5-1 nM were used with 7 concentrations of the competing drug pre-dissolved in DMSO and diluted in buffer. The assays were incubated for 1-3 hours at room temperature, and terminated by rapid filtration through Whatman GF/B filters pretreated with 0.3% polyethyleneimine using a Brandel cell harvester. The radioactivity was determined in a Packard Tri-Carb 2900TR liquid scintillation counter. Data were analyzed by non-linear regression analyses using PRISM 4.00 (GraphPad Software Inc., San Diego, Calif.).

More information about the assay can be found in Hirst, W. D., Minton, J. A. L., Bromidge, S. M., Moss, S. F., Latter, A., Riley, G., Routledge, C., Middlemiss, D. N. & Price, G. W. (2000). Characterization of [¹²⁵I]-SB-258585 binding to human recombinant and native 5-HT6 receptors in rat, pig and human brain tissue is described in Br. J. Pharmacol., 130, 1597-1605.

Results

Typical IC₅₀ values as measured in the assays described above are 5 μM or less. In one aspect of the invention the IC₅₀ is below 500 nM. In another aspect of the invention the IC₅₀ is below 50 nM. In a further aspect of the invention the IC₅₀ is below 10 nM.

TABLE 1
Specimen results from assay.

| Example no | Kᵢ (nM) | n |
|---|---|---|
| 6 | 31 ± 9 | 3 |
| 39 | 32 ± 14 | 2 |

1. A compound having the formula I 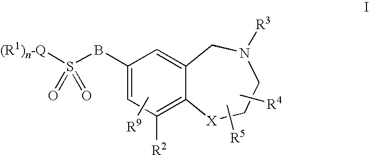
wherein:
Q is C₆₋₁₀arylC₀₋₆alkyl, C₅₋₁₁heteroarylC₀₋₆alkyl, C₃₋₄cycloalkylC₀₋₆alkyl, C₃₋₇heterocycloalkylC₀₋₆alkyl or C₁₋₁₀alkyl;
R¹ is hydrogen, hydroxyl, halogen, C₁₋₁₀alkyl, C₂₋₁₀alkenyl, C₂₋₁₀alkenyl, C₁₋₁₀alkoxy, N(R¹⁰)₂, C₆₋₁₀arylC₀₋₆alkyl, C₅₋₆heteroarylC₀₋₆alkyl, C₁₋₆haloalkyl, C₁₋₆haloalkylO, R⁸OC₀₋₆alkyl, CN, SR⁷, R⁷SO₂C₀₋₆alkyl, SO₂R⁷, R⁷CON(R⁸)C₀₋₆alkyl, NR⁸SO₂R⁷, COR⁷, COOR⁸, OSO₂R⁸, (R⁸)₂NCOC₀₋₆alkyl, SO₂N(R⁸)₂, N(R⁸)CON(R⁸)₂, NO₂, C₃₋₆cycloalkyl, C₃₋₆heterocycloalkyl or oxo;
n is 0, 1, 2, 3, 4 or 5;
B is O, N(R⁶)₂, or B is NR⁶ within a C₅₋₁₁heteroaryl wherein R⁶ forms a ring with Q;
X is O, CH₂, CO, S, SO, SO₂ or NR¹²;
R² is hydrogen, hydroxyl, halogen, C₁₋₁₀alkyl, C₂₋₁₀oalkenyl, C₂₋₁₀alkynyl, C₁₋₁₀alkoxy, N(R¹⁰)₂, C₆₋₁₀arylC₀₋₆alkyl, C₅₋₆heteroarylC₀₋₆alkyl, C₁₋₆haloalkyl, C₁₋₆haloalkylO, R⁷OC₀₋₆alkyl, CN, SR⁷, SO₂R⁸, SOR⁷, N(R⁸)COR⁷, N(R⁸)SO₂R⁷, COR⁷, COOR⁷, OSO₂R⁷, CON(R⁸)₂ or SO₂N(R⁸)₂;
R³ is hydrogen, C₁₋₁₀alkyl, C₂₋₁₀alkenyl, C₂₋₁₀alkynyl, C₆₋₁₀arylC₀₋₆alkyl, C₅₋₆heteroarylC₀₋₆alkyl, C₁₋₆haloalkyl or R⁷OC₁₋₆alkyl;
R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy; or
R³ and R⁴ form together a C₃₄heterocycloalkyl, and which may be substituted by one or more groups selected independently from hydrogen, halogen, C₁₋₆alkyl, C₁₋₆haloalkyl, COR¹¹, SO₂R¹¹, OR¹¹, cyano, oxo and SO₂N(R¹⁰)₂;
R⁵ is hydrogen, C₁₋₆alkyl, C₁₋₆alkoxy, C₁₋₆haloalkoxy or C₁₋₆haloalkyl; or
R⁴ and R⁵ form together a C₃₋₇heterocycloalkyl or a C₃₋₇cycloalkyl, and which may be substituted by one or more groups selected independently from hydrogen, halogen, C₁₋₆alkyl, C₁₋₆haloalkyl, COR¹¹, SO₂R¹¹, OR¹¹, cyano, oxo and SO₂N(R¹⁰)₂;
R⁶ is hydrogen, C₁₋₆alkyl, C₃₋₆cycloakylC₀₋₆alkyl, R⁷OC₁₋₆alkyl, C₁₋₆haloalkyl, C₁₋₆cyanoalkyl, (R¹⁰)₂NCOC₀₋₆alkyl or R¹¹SO₂C₁₋₆alkyl;
R⁷ is C₁₋₆alkyl, C₆₋₁₀arylC₀₋₆alkyl, C₅₋₆heteroarylC₀₋₆alkyl, C₃₋₇cycloalkylC₀₋₆alkyl or C₁₋₆haloalkyl;
R⁸ is hydrogen, C₁₋₆alkyl, C₁₋₆haloalkyl, C₃₋₇cycloalkylC₀₋₆alkyl, C₆₋₁₀arylC₀₋₆alkyl or C₅₋₆heteroarylC₀₋₆alkyl; or
R⁷ and R⁸ form together a C₅₋₆heteroaryl or C₃₋₇heterocycloalkyl;
whereby any aryl and heteroaryl under R¹, R³, R⁷ and R⁸ may be substituted by one or more groups selected independently from hydrogen, halogen, hydroxyl, C₁₋₆haloalkyl, CN, OR¹⁰, C₁₋₆alkyl, oxo, SR¹⁰, CON(R¹⁰)₂, N(R¹⁰)COR¹¹, SO₂R¹¹, SOR¹¹, N(R¹⁰)₂ and COR¹¹;
R⁹ is hydrogen, hydroxyl, halogen, C₁₋₆alkyl, C₁₋₆alkoxyC₀₋₃alkyl, C₁₋₆haloalkyl, COR¹¹, CON(R¹⁰)₂, N(R¹⁰)COR¹¹, SR¹⁰SOR¹¹, CN or SO₂R¹¹;
R¹⁰ is hydrogen, C₁₋₆alkyl or C₁₋₆haloalkyl;
R¹¹ is C₁₋₆alkyl or C₁₋₆haloalkyl; or
R¹⁰ and R¹¹ form together a C₃₋₇heterocycloalkyl, which may be substituted by one or more groups selected independently from hydrogen, halogen, hydroxyl, C₁₋₃alkyl, C₁₋₃alkoxy and cyano; and
R¹² is hydrogen, C₁₋₆alkyl, C₁₋₆haloalkyl, COR¹¹ or SO₂R¹¹;
or salts, solvates or solvated salts thereof.

2. The compound according to claim 1, wherein:
Q is C₆₋₁₀arylC₀₋₆alkyl, C₅₋₁₁heteroarylC₀₋₆alkyl, C₃₋₇cycloalkylC₀₋₆alkyl, C₃₋₇heterocycloalkylC₀₋₆alkyl or C₁₋₁₀alkyl;
R¹ is hydrogen, hydroxyl, halogen, C₁₋₁₀alkyl, C₂₋₁₀alkenyl, C₂₋₁₀alkynyl, C₁₋₁₀alkoxy, N(R¹⁰)₂, C₆₋₁₀arylC₀₋₆alkyl, C₅₋₆heteroarylC₀₋₆alkyl, C₁₋₆haloalkyl, C₁₋₆haloalkylO, R⁸OC₀₋₆alkyl, CN, SR⁷, R⁷SO₂C₀₋₆alkyl, SO₂R⁷, R⁷CON(R⁸)C₀₋₆alkyl, NR⁸SO₂R⁷, COR⁷, COOR⁸, OSO₂R⁸, (R⁸)₂NCOC₀₋₆alkyl, SO₂N(R⁸)₂, N(R⁸)CON(R⁸)₂, NO₂, C₃₋₆cycloalkyl, C₃₋₆heterocycloalkyl or oxo;
n is 0, 1 or 2;
B is O or N(R⁶)₂;
X is O or CH₂;
R² is hydrogen, hydroxyl, halogen, C₁₋₁₀alkyl, C₁₋₁₀alkoxy, C₆₋₁₀arylC₀₋₆alkyl, C₅₋₆heteroarylC₀₋₆alkyl or C₁₋₆haloalkyl;
R³ is hydrogen, C₁₋₁₀alkyl, C₆₋₁₀arylC₀₋₆alkyl, C₅₋₆heteroarylC₀₋₆alkyl, C₁₋₆haloalkyl or R⁷OC₁₋₆alkyl;
R⁴ is hydrogen, C₁₋₅alkyl, C₁₋₅haloalkyl, C₁₋₅alkoxy or C₁₋₅haloalkoxy and may be substituted by one or more groups selected independently from halogen, hydroxyl, cyano, C₁₋₃alkyl and C₁₋₃alkoxy; or
R³ and R⁴ form together a C₃₋₇heterocycloalkyl, and which may be substituted by one or more groups selected independently from hydrogen, halogen, C₁₋₆alkyl, C₁₋₆haloalkyl, COR¹¹, SO₂R¹¹, OR¹¹, cyano, oxo and SO₂N(R¹⁰)₂;
R⁵ is hydrogen;
R⁶ is hydrogen, C₁₋₆alkyl, C₃₋₆cycloakylC₀₋₆alkyl, R⁷OC₁₋₆alkyl, C₁₋₆haloalkyl or C₁₋₆cyanoalkyl;
R⁷ is C₁₋₆alkyl, C₆₋₁₀arylC₀₋₆alkyl, C₅₋₆heteroarylC₀₋₆alkyl, C₃₋₇cycloalkylC₀₋₆alkyl or C₁₋₆haloalkyl;
R⁸ is hydrogen, C₁₋₆alkyl, C₁₋₆haloalkyl, C₃₋₇cycloalkylC₀₋₆alkyl, C₆₋₁₀arylC₀₋₆alkyl or C₅₋₆heteroarylC₀₋₆alkyl;
whereby any aryl and heteroaryl under R¹ may be substituted by one or more groups selected independently from hydrogen, halogen, hydroxyl, C₁₋₆haloalkyl, CN, OR¹⁰, C₁₋₆alkyl, oxo, SR¹⁰, CON(R¹⁰)₂, N(R¹⁰)COR¹¹, SO₂R¹¹, SOR¹¹, N(R¹⁰)₂ and COR¹¹; and
R¹⁰ is hydrogen, C₁₋₆alkyl or C₁₋₆haloalkyl;
or salts, solvates or solvated salts thereof.

3. The compound according to any one of claims 1 to 2, wherein Q is C₆₋₁₀arylC₀₋₄alkyl or C₅₋₁₁heteroarylC₀₋₄alkyl.

4. The compound according to any one of claims 1 to 3, wherein Q is phenyl, naftyl, benzothienyl, thiazole, pyrrolyl, pyridinyl, benzofuranyl, quinolinyl, phenylmethyl, tetralinyl, imidazothiazole or thienyl.

5. The compound according to any one of claims 1 to 4, wherein R¹ is hydrogen, halogen, C₁₋₄alkyl, C₁₋₄alkoxy, C₆₋₁₀arylC₀₋₄alkyl, C₅₋₆heteroarylC₀₋₄alkyl, C₁₋₄haloalkyl, COR⁷, R⁸OC₀₋₄alkyl, SO₂R⁷ or R⁷CON(R⁸)C₀₋₄alkyl.

6. The compound according to any one of claims 1 to 5, wherein B is N(R⁶)₂, and R⁶ is hydrogen or C₁₋₃alkyl.

7. The compound according to any one of claims 1 to 6, wherein R² is hydrogen, halogen or C₁₋₄haloalkyl.

8. The compound according to any one of claims 1 to 7, wherein R³ is hydrogen, C₁₋₃alkyl or C₁₋₄haloalkyl.

9. The compound according to any one of claims 1 to 8, wherein R³ and R⁴ form together a C₃₋₆heterocycloalkyl.

10. Compounds according to claim 1 selected from the group consisting of:
3-bromo-N-(4-methyl-2,3,4,5-tetrahydro-1,4-benzoxazepin-7-yl)benzenesulfonamide;
N-(4-methyl-2,3,4,5-tetrahydro-1,4-benzoxazepin-7-yl)benzenesulfonamide;
2-chloro-N-(4-methyl-2,3,4,5-tetrahydro-1,4-benzoxazepin-7-yl)benzenesulfonamide;
3,5-dichloro-N-(4-methyl-2,3,4,5-tetrahydro-1,4-benzoxazepin-7-yl)benzenesulfonamide;
4-chloro-N-(4-methyl-2,3,4,5-tetrahydro-1,4-benzoxazepin-7-yl)benzenesulfonamide;
2,3-dichloro-N-(4-methyl-2,3,4,5-tetrahydro-1,4-benzoxazepin-7-yl)benzenesulfonamide;
3-methoxy-N-(4-methyl-2,3,4,5-tetrahydro-1,4-benzoxazepin-7-yl)benzenesulfonamide;
3-bromo-N-(9-chloro-4-isopropyl-2,3,4,5-tetrahydro-1,4-benzoxazepin-7-yl)benzenesulfonamide;
2,3-dichloro-N-(2,3,4,5-tetrahydro-1,4-benzoxazepin-7-yl)benzenesulfonamide;
4-chloro-N-(2,3,4,5-tetrahydro-1,4-benzoxazepin-7-yl)naphthalene-1-sulfonamide;
4-fluoro-N-(2,3,4,5-tetrahydro-1,4-benzoxazepin-7-yl)naphthalene-1-sulfonamide;
3-chloro-2-fluoro-N-(2,3,4,5-tetrahydro-1,4-benzoxazepin-7-yl)benzenesulfonamide;
5-chloro-N-(2,3,4,5-tetrahydro-1,4-benzoxazepin-7-yl)naphthalene-2-sulfonamide;
5-chloro-N-(2,3,4,5-tetrahydro-1,4-benzoxazepin-7-yl)naphthalene-1-sulfonamide;
N-(2,3,4,5-tetrahydro-1,4-benzoxazepin-7-yl)biphenyl-2-sulfonamide;
N-(2,3,4,5-tetrahydro-1,4-benzoxazepin-7-yl)biphenyl-3-sulfonamide;
N-(2,3,4,5-tetrahydro-1,4-benzoxazepin-7-yl)-2,3-dihydro-1-benzofuran-5-sulfonamide;
N-(2,3,4,5-tetrahydro-1,4-benzoxazepin-7-yl)quinoline-8-sulfonamide;
1-(3-chlorophenyl)-N-(2,3,4,5-tetrahydro-1,4-benzoxazepin-7-yl)methanesulfonamide;
N-(2,3,4,5-tetrahydro-1,4-benzoxazepin-7-yl)-5,6,7,8-tetrahydronaphthalene-2-sulfonamide;
N-(2,3,4,5-tetrahydro-1,4-benzoxazepin-7-yl)-5,6,7,8-tetrahydronaphthalene-1-sulfonamide;
2,3-dichloro-N-[4-(2-fluoroethyl)-2,3,4,5-tetrahydro-1,4-benzoxazepin-7-yl]benzenesulfonamide;
4-chloro-N-[4-(2-fluoroethyl)-2,3,4,5-tetrahydro-1,4-benzoxazepin-7-yl]naphthalene-1-sulfonamide;
2,3-dichloro-N-[(11aS)-2,3,11,11a-tetrahydro-1H,5H-pyrrolo[2,1-c][1,4]benzoxazepin-7-yl]benzenesulfonamide;
N-[(11aS)-2,3,11,11a-tetrahydro-1H,5H-pyrrolo[2,1-c][1,4]benzoxazepin-7-yl]naphthalene-1-sulfonamide;
N-[(11aS)-2,3,11,11a-tetrahydro-1H,5H-pyrrolo[2,1-c][1,4]benzoxazepin-7-yl]biphenyl-4-sulfonamide;
N-[(11aS)-2,3,11,11a-tetrahydro-1H,5H-pyrrolo[2,1-c][1,4]benzoxazepin-7-yl]-3-(trifluoromethyl)benzenesulfonamide;
3-bromo-N-[9-(trifluoromethyl)-2,3,4,5-tetrahydro-1,4-benzoxazepin-7-yl]benzenesulfonamide;
5-chloro-3-methyl-N-[9-(trifluoromethyl)-2,3,4,5-tetrahydro-1,4-benzoxazepin-7-yl]-1-benzothiophene-2-sulfonamide;
N-[4-methyl-5-({[9-(trifluoromethyl)-2,3,4,5-tetrahydro-1,4-benzoxazepin-7-yl]amino}sulfonyl)-1,3-thiazol-2-yl]acetamide;
2,3-dichloro-N-[9-(trifluoromethyl)-2,3,4,5-tetrahydro-1,4-benzoxazepin-7-yl]benzenesulfonamide;
3-(trifluoromethyl)-N-[9-(trifluoromethyl)-2,3,4,5-tetrahydro-1,4-benzoxazepin-7-yl]benzenesulfonamide;
N-[9-(trifluoromethyl)-2,3,4,5-tetrahydro-1,4-benzoxazepin-7-yl]naphthalene-1-sulfonamide;
2,3-dichloro-N-(2,3,4,5-tetrahydro-1H-2-benzazepin-8-yl)benzenesulfonamide;
4-chloro-N-(2,3,4,5-tetrahydro-1H-2-benzazepin-8-yl)naphthalene-1-sulfonamide;
N-(2,3,4,5-tetrahydro-1H-2-benzazepin-8-yl)biphenyl-2-sulfonamide;
N-(2,3,4,5-tetrahydro-1H-2-benzazepin-8-yl)quinoline-8-sulfonamide;
1-[3-chloro-5-(trifluoromethyl)pyridin-2-yl]-N-(2,3,4,5-tetrahydro-1H-2-benzazepin-8-yl)-1H-pyrrole-2-sulfonamide;
4-phenyl-N-(2,3,4,5-tetrahydro-1H-2-benzazepin-8-yl)-5-(trifluoromethyl)thiophene-3-sulfonamide;
4′-chloro-N-(2,3,4,5-tetrahydro-1H-2-benzazepin-8-yl)biphenyl-2-sulfonamide;
4-(phenylsulfonyl)-N-(2,3,4,5-tetrahydro-1H-2-benzazepin-8-yl)thiophene-2-sulfonamide;
5-chloro-3-methyl-N-(2,3,4,5-tetrahydro-1H-2-benzazepin-8-yl)-1-benzothiophene-2-sulfonamide;
2-bromo-N-(2,3,4,5-tetrahydro-1H-2-benzazepin-8-yl)benzenesulfonamide;
N-(2,3,4,5-tetrahydro-1H-2-benzazepin-8-yl)-2-(trifluoromethyl)benzenesulfonamide;
2-iodo-N-(2,3,4,5-tetrahydro-1H-2-benzazepin-8-yl)benzenesulfonamide;
2,6-dichloro-N-(2,3,4,5-tetrahydro-1H-2-benzazepin-8-yl)benzenesulfonamide;
N-(2,3,4,5-tetrahydro-1H-2-benzazepin-8-yl)-2-(trifluoromethoxy)benzenesulfonamide;
3,4-dichloro-N-(2,3,4,5-tetrahydro-1H-2-benzazepin-8-yl)benzenesulfonamide;
N-(2,3,4,5-tetrahydro-1H-2-benzazepin-8-yl)biphenyl-4-sulfonamide;
2,3-dichloro-N-(2-methyl-2,3,4,5-tetrahydro-1H-2-benzazepin-8-yl)benzenesulfonamide;
4-chloro-N-(2-methyl-2,3,4,5-tetrahydro-1H-2-benzazepin-8-yl)naphthalene-1-sulfonamide;
2-benzoyl-4-chloro-N-(2,3,4,5-tetrahydro-1H-2-benzazepin-8-yl)benzenesulfonamide; and
6-chloro-N-(2,3,4,5-tetrahydro-1H-2-benzazepin-8-yl)imidazo[2,1-b][1,3]thiazole-5- or salts, solvates or solvated salts thereof.

11. The compound according to any one of claims 1 to 10, for use in therapy.

12. Use of the compounds of formula I according to any one of claims 1 to 10, in the manufacture of a medicament for treatment of 5-HT6 mediated disorders.

13. Use of the compounds of formula I according to any one of claims 1 to 10, in the manufacture of a medicament for treatment of Alzheimer's disease, cognitive disorders, cognitive impairment associated with schizophrenia, obesity and/or Parkinson's disease.

14. A pharmaceutical composition comprising as active ingredient a therapeutically effective amount of the compound according to any one of claims 1 to 10, in association with one or more pharmaceutically acceptable diluents, excipients and/or inert carriers.

15. The pharmaceutical composition according to claim 14, for use in the treatment of 5-HT6 mediated disorders.

16. A method of treatment of 5-HT6 mediated disorders, comprising administering to a mammal, including man in need of such treatment, a therapeutically effective amount of the compounds of formula I, according to any one of claims 1 to 10.

17. An agent for the treatment of 5-HT6 mediated disorders, which comprises as active ingredient a compound of formula I, according to any one of claims 1 to 10.

18. Compounds of formula II 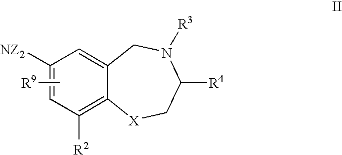
wherein Z is N or O, and wherein X, R², R³, R⁴ and R⁹ are defined as in claim 1.

19. The use of the compound according to claim 18 in the preparation of compounds of formula I according to claim 1.
